(19) United States
(12) Patent Application Publication
(10) Pub. No.: US 2023/0148881 A1
(43) Pub. Date: May 18, 2023

(54) SKIN CONDITION ESTIMATION METHOD, SKIN CONDITION ESTIMATION DEVICE, AND SKIN CONDITION ESTIMATION SYSTEM

(71) Applicant: Murata Manufacturing Co., Ltd., Kyoto-fu (JP)

(72) Inventors: Rie YAMANE, Nagaokakyo-shi (JP); Tomonori SAKAMOTO, Nagaokakyo-shi (JP); Yuuji NIHEI, Nagaokakyo-shi (JP)

(21) Appl. No.: 18/156,694

(22) Filed: Jan. 19, 2023

Related U.S. Application Data
(63) Continuation of application No. PCT/JP2021/027725, filed on Jul. 27, 2021.

(30) Foreign Application Priority Data
Aug. 3, 2020 (JP) 2020-131861

(51) Int. Cl. A61B 5/021; A61B 5/00; G16H 50/30

(52) U.S. Cl. CPC A61B 5/02108; A61B 5/68; G16H 50/30

(57) ABSTRACT

A skin condition estimation method of the present disclosure is a skin condition estimation method executed by a computer and includes: acquiring first information related to hormone balance; and estimating a future skin condition on an estimation date after a first acquisition date on which the first information is acquired based on the first information.

### CROSS REFERENCE TO RELATED APPLICATION

This is a continuation application of International Application No. PCT/JP2021/027725, with an international filing date of Jul. 27, 2021, which claims priority of Japanese Patent Application No. 2020-131861 filed on Aug. 3, 2020, the contents of which are incorporated herein by reference.

### BACKGROUND ART

The present disclosure relates to a skin condition estimation method, a skin condition estimation device, and a skin condition estimation system.

JP2020-14710A discloses a skin condition differentiation method for estimating a skin condition using a muscle amount as an index. In the differentiation method described in JP2020-14710A, analysis is performed by deriving a parameter representing a skin condition by applying a muscle amount obtained by measurement to an estimation equation obtained by multivariate analysis.

### BRIEF SUMMARY

In recent years, it has become desirable to estimate a future skin condition.

A skin condition estimation method according to an aspect of the present disclosure is a skin condition estimation method executed by a computer and includes: acquiring first information related to hormone balance; and estimating a future skin condition on an estimation date after a first acquisition date on which the first information is acquired based on the first information.

A skin condition estimation device according to an aspect of the present disclosure includes: a measurement unit that acquires information related to hormone balance; and an estimator that estimates future skin information on an estimation date after a date on which the information is acquired based on the information acquired by the measurement unit.

A skin condition estimation system according to an aspect of the present disclosure includes: a measurement device; and a processing device that communicates with the measurement device, in which the measurement device includes: a measurement unit that acquires information related to hormone balance; and a first communicator that transmits the information, and in which the processing device includes: a second communicator that receives the information; and an estimator that estimates a future skin condition on an estimation date after a date on which the information is acquired based on the information.

According to the present disclosure, a future skin condition can be estimated.

### BRIEF DESCRIPTION OF DRAWINGS

FIG. 1 is a block diagram showing a schematic configuration of an example of a skin condition estimation device according to a first embodiment of the present disclosure;

FIG. 2 is a schematic diagram showing an example of measurement timing;

FIG. 3 is a graph showing an example of a correlation coefficient between a basal body temperature and a skin condition;

FIG. 4 is a flowchart showing an example of a skin condition estimation method according to the first embodiment of the present disclosure;

FIG. 5 is a block diagram showing a schematic configuration of a skin condition estimation device according to a first modification of the first embodiment of the present disclosure;

FIG. 6A is a schematic view showing an example of a display screen of a display unit according to the first modification;

FIG. 6B is a schematic view showing another example of the display screen of the display unit according to the first modification;

FIG. 7 is a block diagram showing a schematic configuration of an example of a skin condition estimation device according to a second embodiment of the present disclosure;

FIG. 8 is a schematic diagram showing an example of an acceleration pulse wave;

FIG. 9 is a schematic diagram showing an example of measurement timing;

FIG. 10 is a flowchart showing an example of a skin condition estimation method according to the second embodiment of the present disclosure;

FIG. 11 is a block diagram showing a schematic configuration of an example of a skin condition estimation device according to a third embodiment of the present disclosure;

FIG. 12 is a schematic diagram showing an example of measurement timing;

FIG. 13 is a flowchart showing an example of a skin condition estimation method according to the third embodiment of the present disclosure;

FIG. 14 is a flowchart of a skin condition estimation method according to a second modification of the third embodiment the present disclosure;

FIG. 15 is a schematic diagram showing an example of measurement timing of the second modification;

FIG. 16 is a block diagram showing a schematic configuration of an example of a skin condition estimation device according to a fourth embodiment of the present disclosure;

FIG. 17 is a flowchart of an example of a machine learning method in a skin condition estimation method according to the fourth embodiment the present disclosure;

FIG. 18 is a block diagram showing a schematic configuration of an example of a skin condition estimation device according to a fifth embodiment of the present disclosure;

FIG. 19 is a block diagram showing a schematic configuration of an example of a skin condition estimation system according to a sixth embodiment of the present disclosure;

FIG. 20 is a flowchart showing an example of a skin condition estimation method according to the sixth embodiment of the present disclosure;

FIG. 21 is a block diagram showing a schematic configuration of an example of a skin condition estimation system according to a third modification of the sixth embodiment of the present disclosure;

FIG. 22 is a block diagram showing a schematic configuration of an example of a skin condition estimation system according to a seventh embodiment of the present disclosure;

FIG. 23 is a block diagram showing a schematic configuration of an example of a skin condition estimation system according to an eighth embodiment of the present disclosure;

FIG. 24 is a block diagram showing a schematic configuration of an example of a skin condition estimation device according to a ninth embodiment of the present disclosure;

FIG. 25 is a flowchart showing an example of a skin condition estimation method according to the ninth embodiment of the present disclosure;

FIG. 26 is a flowchart of a skin condition estimation method according to a fourth modification of the ninth embodiment of the present disclosure;

FIG. 27 is a graph showing an example of a correlation between an actual measurement value and examples 1 and 2;

FIG. 28 is a graph showing an example of a correlation between a comparative example 1 and an actual measurement value;

FIG. 29 is a graph showing an example of a correlation between a comparative example 2 and an actual measurement value;

FIG. 30 is a graph showing an example of a correlation between a comparative example 3 and an actual measurement value;

FIG. 31 is a graph showing an example of a correlation between a comparative example 4 and an actual measurement value;

FIG. 32 is a table showing an example of correlation coefficients of examples 1 to 3 and the comparative examples 1 to 4;

FIG. 33 is a graph showing an example of a correlation between an actual measurement value and examples 4 and 5;

FIG. 34 is a graph showing an example of a correlation between an actual measurement value and examples 6 and 7;

FIG. 35 is a graph showing an example of a correlation between an actual measurement value and examples 8 and 9; and

FIG. 36 is a table showing an example of correlation coefficients of examples 4 to 9.

### DETAILED DESCRIPTION

### Circumstances Leading to Present Disclosure

In recent years, it has become desirable to estimate a future skin condition. By knowing the future skin condition, effective care for the skin can be performed, and the skin condition can be kept good.

The skin condition differentiation method described in JP2020-14710A estimates the skin condition using the muscle amount as an index. However, in the differentiation method described in JP2020-14710A, although the current skin condition can be estimated, there is a problem that the future skin condition cannot be estimated.

As a result of intensive studies, the present inventors have found that there is a correlation between information related to hormone balance and skin condition. Therefore, the present inventors have found a configuration for acquiring information related to hormone balance and estimating a skin condition in the future from the acquisition date of the information based on the information, thereby achieving the present disclosure.

A skin condition estimation method according to an aspect of the present disclosure is a skin condition estimation method executed by a computer and includes: acquiring first information related to hormone balance; and estimating a future skin condition on an estimation date after a first acquisition date on which the first information is acquired based on the first information.

With such a configuration, the future skin condition can be estimated.

The first information may include at least one piece of information of basal body temperature, brain waves, blood, saliva, and urine.

With such a configuration, the future skin condition can be easily estimated.

The first acquisition date may be 7 days or more and 13 days or less before the estimation date.

With such a configuration, the estimation accuracy of the future skin condition can be improved.

The acquiring the first information may include acquiring a plurality of pieces of the first information on a plurality of different days, and the estimating may include estimating the future skin condition based on the plurality of pieces of the first information acquired on the plurality of different days.

With such a configuration, the estimation accuracy of the future skin condition can be improved.

The skin condition estimation method may further include acquiring second information related to a blood vessel condition, and the estimating may include estimating the future skin condition on the estimation date after a second acquisition date on which the second information is acquired based on the first information and the second information.

With such a configuration, the estimation accuracy of the future skin condition can be improved.

The second information may include at least one piece of information of a pulse wave, a blood pressure, and a form and a function of a blood vessel.

With such a configuration, the future skin condition can be easily estimated.

The second acquisition date may be different from the first acquisition date.

With such a configuration, the estimation accuracy of the future skin condition can be improved.

The second acquisition date may be later than the first acquisition date.

With such a configuration, the estimation accuracy of the future skin condition can be further improved.

The acquiring the second information may include acquiring a plurality of pieces of the second information on a plurality of different days, and the estimating may include estimating the future skin condition based on the plurality of pieces of the second information acquired on the plurality of different days.

With such a configuration, the estimation accuracy of the future skin condition can be further improved.

The skin condition estimation method may further include acquiring third information related to a blood vessel condition different from the second information, and the estimating may include estimating the future skin condition on the estimation date after a third acquisition date on which the third information is acquired based on the first information and the third information.

With such a configuration, the estimation accuracy of the future skin condition can be improved.

The third information may be different from the second acquisition date.

With such a configuration, the future skin condition can be easily estimated.

The third acquisition date may be later than the second acquisition date.

With such a configuration, the estimation accuracy of the future skin condition can be improved.

The acquiring the third information may include acquiring a plurality of pieces of the third information on a plurality of different days, and the estimating may include estimating the future skin condition based on the plurality of pieces of the third information acquired on the plurality of different days.

With such a configuration, the estimation accuracy of the future skin condition can be improved.

The estimation date may be current, and the estimating may include estimating a current skin condition based on the first information acquired in the past.

With such a configuration, the current skin condition can be estimated.

The skin condition estimation method may further include acquiring fourth information having at least one of information related to a current hormone balance and information related to a current blood condition, and the estimating the current skin condition may include estimating the current skin condition based on the first information acquired in the past and the fourth information acquired at present.

With such a configuration, the estimation accuracy of the current skin condition can be improved.

The skin condition estimation method may further include: acquiring actual measurement information of a skin condition; and creating a regression model in which the first information is input and the future skin condition is output using the first information and information of the skin condition as teacher data.

With such a configuration, the estimation accuracy of the future skin condition can be improved.

The estimating may include estimating the future skin condition by inputting the first information to the regression model.

With such a configuration, the estimation accuracy of the future skin condition can be improved.

A skin condition estimation device according to an aspect of the present disclosure includes: a measurement unit that acquires information related to hormone balance; and an estimator that estimates future skin information on an estimation date after a date on which the information is acquired based on the information acquired by the measurement unit.

With such a configuration, the future skin condition can be estimated.

A skin condition estimation system according to an aspect of the present disclosure includes: a measurement device; and a processing device that communicates with the measurement device, in which the measurement device includes: a measurement unit that acquires information related to hormone balance; and a first communicator that transmits the information, and in which the processing device includes: a second communicator that receives the information; and an estimator that estimates a future skin condition on an estimation date after a date on which the information is acquired based on the information.

With such a configuration, the future skin condition can be estimated.

Hereinafter, an embodiment of the present disclosure will be described with reference to the accompanying drawings. Note that the following description is merely exemplary in nature and is not intended to limit the present disclosure, an object for application, or a usage. Furthermore, the drawings are schematic, and ratios of dimensions and the like do not necessarily match actual ones.

### First Embodiment

### Overall Configuration

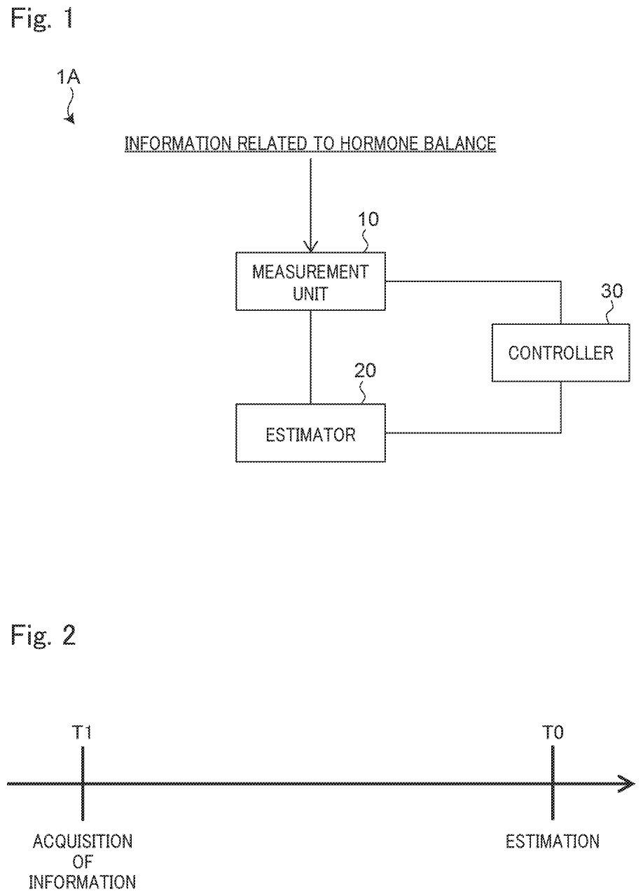

FIG. 1 is a block diagram showing a schematic configuration of an example of a skin condition estimation device 1A according to a first embodiment of the disclosure. As shown in FIG. 1, the skin condition estimation device 1A includes a measurement unit 10, an estimator 20, and a controller 30. The skin condition estimation device 1A estimates a future skin condition based on information measured by the measurement unit 10. In the first embodiment, the skin condition region estimated by the skin condition estimation device 1A is the face of a human.

In the present specification, the “skin condition” includes at least one of pore, wrinkle, texture, pigmented skin color, moisture, and oil content. The “pore” is an evaluation item indicating the conspicuousness of the outlet of the hair protruding from the skin surface. The “wrinkle” is an evaluation item indicating a fold, a crimp, or a ridge formed on the surface of the skin. The “texture” is an evaluation item indicating the beauty of the skin determined by fine irregularities engraved in the skin surface. The “pigmentation” is an evaluation item indicating, for example, a spot or uneven skin color caused by deposition of pigments such as melanin on the skin. The “skin color” is an evaluation item indicating the color tone and brightness of the skin. The “moisture” means the amount of moisture contained in the skin. The “oil content” means the amount of oil contained in the skin. These evaluation items are numerically displayed, for example. For example, the skin condition is expressed by five-grade evaluation. The five-grade evaluation is represented by a numerical range of 1 to 5, and the larger the numerical value, the better.

The skin condition estimation device 1A will be described in detail.

### Measurement Unit

The measurement unit 10 acquires information related to hormone balance. The measurement unit 10 may be referred to as a first measurement unit 10.

The hormone includes, for example, female hormone and male hormone. The female hormone has, for example, estrogen and progesterone. The male hormone has, for example, steroid hormone such as testosterone.

The information related to hormone balance means information correlated with hormone balance. That is, the information related to hormone balance means information capable of estimating a change in hormone balance. For example, the information related to hormone balance is biological information that changes with a change in the amount of hormone secreted. For example, the information related to hormone balance includes at least one piece of information of basal body temperature, brain waves, blood, saliva, and urine.

In the present specification, the information related to hormone balance may be referred to as first information.

The “basal body temperature” is a body temperature measured in a resting state in which factors such as a body temperature change due to activity are excluded and only minimum energy necessary for life support is consumed. For example, the basal body temperature can be measured by a basal thermometer in a resting state at the time of awakening. The basal body temperature is expressed in 0.01 units (second decimal place).

The “brain waves” are brain waves measured by an electroencephalograph. Examples of the brain waves related to hormone balance include brain waves around 20 or more and 22 Hz or less, 11 Hz, 14 Hz, and 8 or more and 10 Hz or less.

The “blood” is, for example, information of the amount of hormone (for example, progesterone) contained in the blood. The amount of hormone contained in the blood can be measured, for example, by a blood test.

The “saliva” is, for example, information of the amount of hormone contained in the saliva or the amount of saliva secreted. The amount of hormone contained in the saliva and the amount of saliva secreted can be measured by, for example, a saliva test. In addition, the amount of saliva secreted can be measured by, for example, an oral wetting meter.

The “urine” is, for example, information of the amount of hormone (for example, estrogen) contained in the urine. The amount of hormone contained in the urine can be measured, for example, by a urinalysis.

The measurement unit 10 is a measurement device (e.g., a sensor) capable of measuring at least one piece of information of basal body temperature, brain waves, blood, saliva, and urine as the information related to hormone balance.

In the first embodiment, an example in which the information related to hormone balance is a basal body temperature will be described. Therefore, the measurement unit 10 measures the basal body temperature. The measurement unit 10 includes, for example, a basal thermometer.

The measurement unit 10 transmits the information related to hormone balance to the estimator 20. Specifically, the measurement unit 10 transmits information of the basal body temperature to the estimator 20. Alternatively, the information of the basal body temperature measured by the measurement unit 10 is input to the estimator 20.

FIG. 2 is a schematic diagram showing an example of measurement timing. In FIG. 2, T0 indicates an estimation date on which the skin condition is estimated. T1 indicates a measurement date of the basal body temperature by the measurement unit 10, that is, an acquisition date of the basal body temperature. As shown in FIG. 2, the measurement unit 10 measures the basal body temperature on the acquisition date T1 before the estimation date T0.

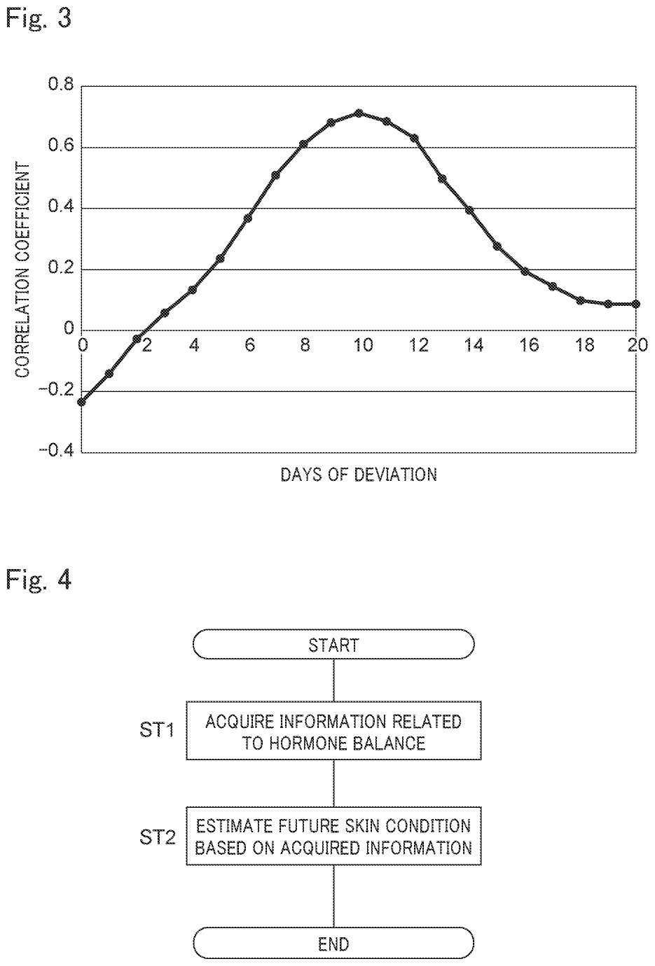

FIG. 3 is a graph showing an example of a correlation coefficient between a basal body temperature and a skin condition. In FIG. 3, the horizontal axis represents the number of days of deviation of the acquisition date T1 of the basal body temperature from the estimation date T0, and the vertical axis represents the correlation coefficient between the basal body temperature and the skin condition. The correlation coefficient indicates the degree of correlation between the basal body temperature and the skin condition. A higher correlation coefficient indicates a higher correlation between the basal body temperature and the skin condition. The correlation coefficient is calculated based on an estimation score of the skin condition estimated based on the basal body temperature and an actual measurement score obtained by actually measuring the skin condition. For example, the correlation coefficient is calculated by dividing the covariance by the standard deviation of the respective variables. The calculation of the estimation score will be described later. The measured score was measured using a measurement device capable of scoring the skin condition. As the measurement device, for example, a skin analysis system “Beauty Explorer (registered trademark)” manufactured by Sony Corporation can be used.

It can be said that the correlation coefficient has a correlation of 0.5 or more. That is, when the correlation coefficient is 0.5 or more, the skin condition can be estimated with high accuracy based on the information of the basal body temperature. As shown in FIG. 3, the acquisition date T1 of the basal body temperature by the measurement unit 10 with the correlation coefficient of 0.5 or more is 7 days or more and 13 days or less before the estimation date T0. The acquisition date T1 of the basal body temperature with the correlation coefficient of 0.6 or more is 8 days or more and 12 days or less before the estimation date T0. In addition, the acquisition date T1 at which the correlation coefficient becomes the highest is a day 10 days before the estimation date T0.

Therefore, the acquisition date T1 by the measurement unit 10 is 7 days or more and 13 days or less before the estimation date T0. Preferably, the acquisition date T1 is 8 days or more and 12 days or less before the estimation date T0. More preferably, the acquisition date T1 is a date 10 days before the estimation date T0. As a result, the estimation accuracy of the skin condition by the estimator 20 can be improved.

### Estimator

The estimator 20 estimates a future skin condition based on the information acquired by the measurement unit 10. Specifically, the estimator 20 estimates the future skin condition on the estimation date T0 after the acquisition date T1 of the information acquired by the measurement unit 10 based on the information acquired by the measurement unit 10.

In the first embodiment, the estimator 20 estimates the future skin condition on the estimation date T0 after the acquisition date T1 when the basal body temperature is acquired based on the basal body temperature acquired by the measurement unit 10.

The estimator 20 estimates a future skin condition within 7 days or more and 13 days or less from the acquisition date T1 of the basal body temperature by the measurement unit 10. Preferably, the estimator 20 estimates a future skin condition within 8 days or more and 12 days or less from the acquisition date T1 of the basal body temperature by the measurement unit 10. More preferably, the estimator 20 estimates a future skin condition 10 days after the acquisition date T1 of the basal body temperature by the measurement unit 10.

In the first embodiment, the estimator 20 estimates the future skin condition on the estimation date T0 on the date of the acquisition date T1 when the measurement unit 10 acquires the information. That is, the acquisition date T1 is current, and the estimation date T0 is later than the acquisition date T1.

The estimator 20 receives information of the basal body temperature from the measurement unit 10. The estimator 20 executes regression analysis using information of the basal body temperature. Specifically, the estimator 20 includes a regression model subjected to machine learning in advance. The regression model is stored in a storage of the estimator 20. In the first embodiment, the regression model is a model in which the information of the basal body temperature is input and the information of the future skin condition is output. The output information of the future skin condition is, for example, information obtained by quantifying the evaluation of the skin condition.

The estimator 20 inputs the information of the basal body temperature measured by the measurement unit 10 to the regression model. The estimator 20 estimates the future skin condition on the estimation date T0 after the acquisition date T1 based on the basal body temperature using the regression model. That is, the estimator 20 outputs the information of the future skin condition by inputting the basal body temperature to the regression model.

The estimator 20 can be implemented by, for example, a semiconductor element or the like. For example, the estimator 20 can include a microcomputer, a central processing unit (CPU), a micro processing unit (MPU), a graphics processing unit (GPU), a digital signal processor (DSP), a field programmable gate array (FPGA), or an application specific integrated circuit (ASIC).

### Controller

The controller 30 integrally controls the components of the skin condition estimation device 1A. The controller 30 includes, for example, a memory that stores a program, and a processing circuit (not shown) corresponding to a processor such as a central processing unit (CPU). In the controller 30, the processor executes the program stored in the memory. The controller 30 can be implemented by, for example, a semiconductor element or the like. For example, the controller 30 may include a microcomputer, a CPU, an MPU, a GPU, a DSP, an FPGA, or an ASIC. The functions of the controller 30 may be configured only by hardware, or may be realized by combining hardware and software. In the first embodiment, the controller 30 controls the measurement unit 10 and the estimator 20.

The skin condition estimation device 1A can be realized by, for example, an information processing device such as a computer. For example, the measurement unit 10, the estimator 20, and the controller 30 may be realized as components of a computer.

### Operation

An example of the operation (skin condition estimation method) of the skin condition estimation device 1A will be described with reference to FIG. 4. FIG. 4 is a flowchart showing an example of the skin condition estimation method according to the first embodiment of the present disclosure. The skin condition estimation method shown in FIG. 4 is executed by the skin condition estimation device 1A.

As shown in FIG. 4, in step ST1, information related to hormone balance is acquired. In step ST1, the measurement unit 10 acquires the information related to hormone balance. Specifically, the measurement unit 10 acquires at least one piece of information of basal body temperature, brain waves, blood, saliva, and urine as the information related to hormone balance. In the first embodiment, the measurement unit 10 measures a basal body temperature.

In step ST2, a future skin condition is estimated based on the information acquired by the measurement unit 10. In step ST2, the estimator 20 estimates the skin condition in the future from the acquisition date T1 of the information by the measurement unit 10 based on the information related to hormone balance acquired by the measurement unit 10. In the first embodiment, the estimator 20 estimates the skin condition in the future from the acquisition date T1 based on the basal body temperature measured by the measurement unit 10.

The estimator 20 executes regression analysis using the regression model based on the information of the basal body temperature. Specifically, the estimator 20 inputs, to the regression model subjected to machine learning in advance, information of the basal body temperature on the acquisition date T1 that is 7 days or more and 13 days or less before the estimation date T0. Preferably, the estimator 20 inputs, to the regression model, information of the basal body temperature on the acquisition date T1 that is 8 days or more and 12 days or less before the estimation date T0. More preferably, the estimator 20 inputs, to the regression model, information of the basal body temperature on the acquisition date T1 that is 10 days before the estimation date T0. As a result, the estimator 20 estimates the skin condition in the future from the acquisition date T1 by the regression model.

### Effects

According to the skin condition estimation method of the first embodiment, the following effects can be obtained.

The skin condition estimation method includes step ST1 of acquiring information related to hormone balance, and step ST2 of estimating a future skin condition on the estimation date T0 after the acquisition date T1 on which the information is acquired based on the acquired information. With such a configuration, the future skin condition can be easily estimated based on the information related to hormone balance.

The information related to hormone balance includes at least one piece of information of basal body temperature, brain waves, blood, saliva, and urine. With such a configuration, the information related to hormone balance can be easily acquired. Furthermore, the future skin condition can be estimated based on the information acquired from the region other than the face. Furthermore, information such as basal body temperature, brain waves, saliva, and urine can be acquired without invading the user.

The acquisition date T1 is 7 days or more and 13 days or less before the estimation date T0. With such a configuration, information having a high correlation between hormone balance and skin condition can be acquired. As a result, the estimation accuracy of the future skin condition can be improved.

The skin condition estimation device 1A includes the measurement unit 10 that acquires information related to hormone balance, and the estimator 20 that estimates future skin information on the estimation date T0 after the acquisition date T1 when the information is acquired based on the information acquired by the measurement unit 10. With such a configuration, the future skin condition can be easily estimated based on the information related to hormone balance.

In the first embodiment, an example in which the measurement unit 10 measures the basal body temperature has been described, but the present disclosure may not be limited thereto. The measurement unit 10 only needs to be able to acquire information related to hormone balance. Furthermore, the information related to hormone balance may be subjected to arbitrary processing before being input to the regression model of the estimator 20.

In the first embodiment, an example in which the skin condition estimation device 1A includes one measurement unit 10 has been described, but the present disclosure may not be limited thereto. The skin condition estimation device 1A may include one or a plurality of measurement units 10. For example, since the skin condition estimation device 1A includes the plurality of measurement units 10, estimation accuracy of the skin condition can be improved. In addition, the plurality of measurement units 10 may acquire different information.

In the skin condition estimation device 1A, the measurement unit 10 is not an essential component. That is, the skin condition estimation device 1A may not include the measurement unit 10. When the skin condition estimation device 1A does not include the measurement unit 10, the information related to hormone balance may be acquired by a separate measurement device that is not included in the skin condition estimation device 1A. The skin condition estimation device 1A may include an input unit that inputs information related to hormone balance instead of the measurement unit 10. The estimator 20 of the skin condition estimation device 1A may estimate the future skin condition based on the information related to hormone balance input to the input unit.

In the first embodiment, an example in which the estimator 20 estimates the skin condition in the future from the acquisition date of the basal body temperature based on the basal body temperature has been described, but the present disclosure may not be limited thereto. The information used for estimating the skin condition may be information related to hormone balance. The estimator 20 may estimate the future skin condition based on information other than the basal body temperature.

An example in which the estimator 20 estimates the future skin condition on the estimation date T0 on the acquisition date T1 when the information is acquired by the measurement unit 10 has been described, but the present disclosure may not be limited thereto. That is, the timing at which the skin condition is estimated by the estimator 20 may not be limited to the acquisition date T1. The timing at which the skin condition is estimated by the estimator 20 may be other than the acquisition date T1. The timing at which the skin condition is estimated by the estimator 20 may be between the acquisition date T1 and the estimation date T0.

In the first embodiment, an example in which the estimator 20 estimates the future skin condition by the regression analysis using the regression model has been described, but the present disclosure may not be limited thereto. The estimator 20 only needs to be able to estimate the future skin condition based on information related to hormone balance. Furthermore, the estimator 20 may estimate the future skin condition using a model other than the regression model.

In the first embodiment, an example in which the measurement unit 10, the estimator 20, and the controller 30 are formed separately has been described, but the present disclosure may not be limited thereto. For example, at least two of the measurement unit 10, the estimator 20, and the controller 30 may be integrated.

In the first embodiment, an example in which the acquisition date T1 is the present, the estimation date T0 is the future, and the estimator 20 estimates the future skin condition on the estimation date T0 on the acquisition date T1 has been described, but the present disclosure may not be limited thereto. For example, the acquisition date T1 may be the past, and the estimation date T0 may be current. In this case, the estimator 20 may estimate the current skin condition based on the first information acquired in the past. With such a configuration, the current skin condition can be estimated.

In the first embodiment, an example in which the skin condition estimation method includes steps ST1 and ST2 has been described, but the skin condition estimation method may not be limited thereto. In the skin condition estimation method, other steps may be added, some steps may be reduced, or a plurality of steps may be performed in one step.

In the first embodiment, the skin condition estimation device and the skin condition estimation method have been described as an example, but the present disclosure is also applicable to a program and a computer-readable recording medium. For example, the program may cause a computer to execute the skin condition estimation method described above. The computer-readable recording medium may store a program for causing a computer to execute the skin condition estimation method described above. The computer-readable recording medium may be, for example, a RAM, a ROM, an EEPROM, a flash memory, or other memory technologies, a CD-ROM, a DVD, or other optical disk storages, or a magnetic cassette, a magnetic tape, a magnetic disk storage, or other magnetic storage device.

### First Modification

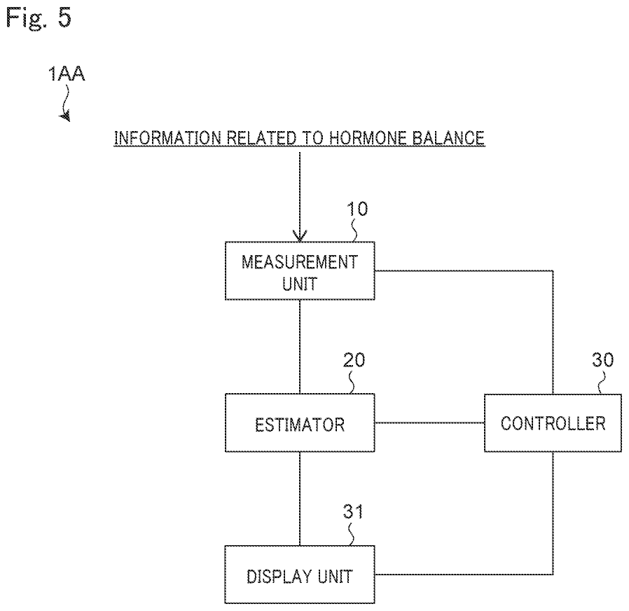

FIG. 5 is a block diagram showing a schematic configuration of a skin condition estimation device 1AA according to a first modification of the first embodiment of the present disclosure. As shown in FIG. 5, the skin condition estimation device 1AA according to the first modification further includes a display unit 31. The display unit 31 displays the estimation result of the skin condition estimated by the estimator 20. The display unit 31 is, for example, a display. The display unit 31 is controlled by the controller 30.

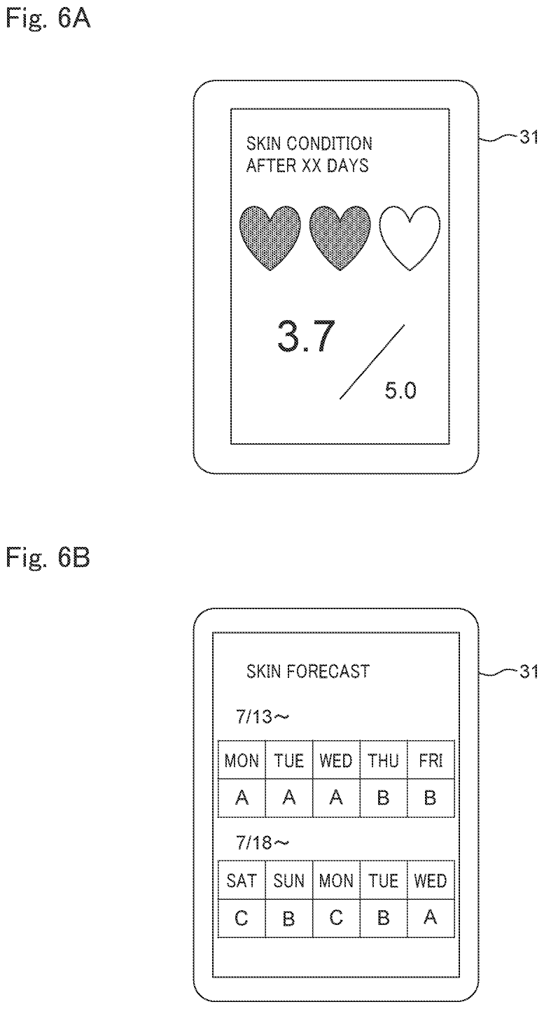

FIG. 6A is a schematic view showing an example of a display screen of the display unit 31 of the first modification. As shown in FIG. 6A, the display unit 31 displays the information of the future skin condition estimated by the estimator 20. The display screen displayed by the display unit 31 includes, for example, the total skin score in the skin condition after XX days. The total score is a numerical value of the comprehensive evaluation of the skin condition, and is indicated by a numerical value in a range of 1 to 5, for example. In addition, an evaluation icon is displayed on the display unit 31 of FIG. 6A in order to visually recognize the total skin score. The evaluation icon is an image in which the total skin score can be visually recognized. The evaluation icons include, for example, a plurality of evaluation icons including colored heart marks and non-colored heart marks. In a case where there are many colored heart marks among the plurality of evaluation icons, it can be recognized that the total skin score is good. Furthermore, in a case where there are many non-colored heart marks among the plurality of evaluation icons, it can be recognized that the total skin score is not good.

FIG. 6B is a schematic diagram showing another example of the display screen of the display unit 31 of the first modification. In FIG. 6B, “A” indicates good, “B” indicates normal, and “C” indicates not good. As shown in FIG. 6B, the display unit 31 may display a skin condition forecast. For example, a skin condition forecast for 10 days may be displayed every day. Specifically, the skin condition forecast may be displayed for each day of the week.

Note that the display screen of the display unit 31 of the first modification may not be limited to the example shown in FIGS. 6A and 6B. In addition to the information shown in FIGS. 6A and 6B, other information may be additionally displayed on the display screen of the display unit 31. Alternatively, the information shown in FIGS. 6A and 6B may be corrected and displayed.

In the modification 1, an example in which the display unit 31 is a display has been described, but the present disclosure may not be limited thereto. For example, the display unit 31 may include one or a plurality of LEDs. The display unit 31 may notify the user whether the future skin condition is good by turning on the LED.

In the modification 1, an example in which the display unit 31 is included in the skin condition estimation device 1AA has been described, but the present disclosure may not be limited thereto. The display unit 31 may not be included in the skin condition estimation device 1AA. The display unit 31 may be a separate body from the skin condition estimation device 1AA. For example, the display unit 31 may be a display screen of an information processing terminal such as a smartphone. The skin condition estimation device 1AA may transmit the estimation result to the display unit 31 of the information processing terminal by network communication or the like. As a result, it is possible to display the estimation result of the future skin condition on the display unit 31 such as an information processing terminal other than the skin condition estimation device 1AA, and thus, it is possible to improve usability.

### Second Embodiment

A skin condition estimation device and a skin condition estimation method according to a second embodiment of the present disclosure will be described. In the second embodiment, points different from the first embodiment will be mainly described. In the second embodiment, the same or equivalent configurations as those of the first embodiment will be described with the same reference numerals. In the second embodiment, the description overlapping with the first embodiment is omitted.

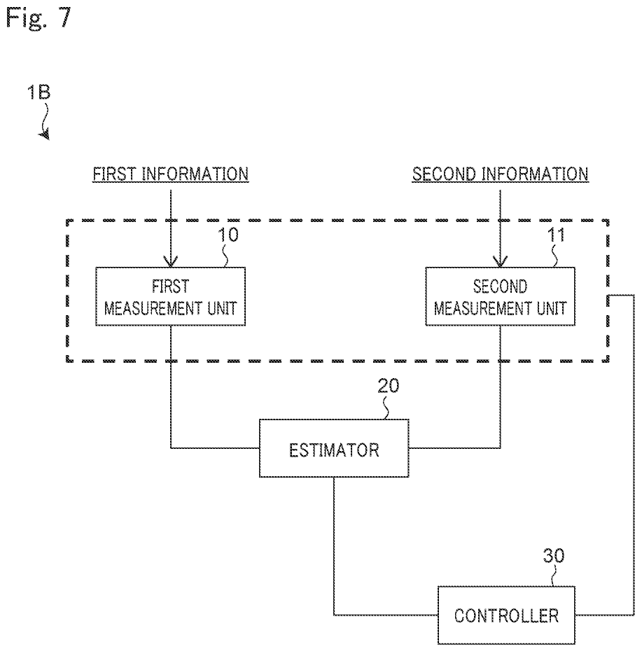

An example of the skin condition estimation device according to the second embodiment will be described with reference to FIG. 7. FIG. 7 is a block diagram showing a schematic configuration of an example of a skin condition estimation device 1B according to the second embodiment of the present disclosure.

The second embodiment is different from the first embodiment in that two measurement units 10 and 11 are provided and the skin condition is estimated based on the information acquired by the two measurement units 10 and 11.

As shown in FIG. 7, the skin condition estimation device 1B further includes the measurement unit 11 that acquires information related to a blood vessel condition. In the second embodiment, the measurement unit 10 is referred to as a first measurement unit 10, and the measurement unit 11 is referred to as a second measurement unit 11. In addition, information related to hormone balance is referred to as first information, and information related to a blood vessel condition is referred to as second information.

### Second Measurement Unit

The second measurement unit 11 acquires the second information related to the blood vessel condition. The second information means information correlated with the blood vessel condition. That is, the second information means information capable of estimating a change in the blood vessel condition. Specifically, the second information includes at least one piece of information of a pulse wave, a blood pressure, and a form and a function of a blood vessel.

The “pulse wave” means a change in volume of a blood vessel that occurs as the heart pumps blood. The pulse wave is measured by, for example, a photoelectric pulse wave sensor. The photoelectric pulse wave sensor is attached to a user's finger to measure a pulse wave, for example.

The “blood pressure” means a pressure of blood applied to a blood vessel wall. The blood pressure is measured by, for example, a sphygmomanometer. Preferably, the sphygmomanometer can measure the maximum blood pressure value and the minimum blood pressure value.

The “form and the function of the blood vessel” is, for example, a thickness of the blood vessel, arteriosclerosis, blood flow, clogging of the blood vessel, and the like. The form and the function of the blood vessels can be measured, for example, by echography.

In the second embodiment, an example in which a pulse wave is used as the second information will be described. The second measurement unit 11 measures a pulse wave. The second measurement unit 11 includes, for example, a photoelectric pulse wave sensor. Specifically, the second information is information of an acceleration pulse wave calculated based on the pulse wave measured by the photoelectric pulse wave sensor. The acceleration pulse wave means a pulse wave differentiated twice on the time axis.

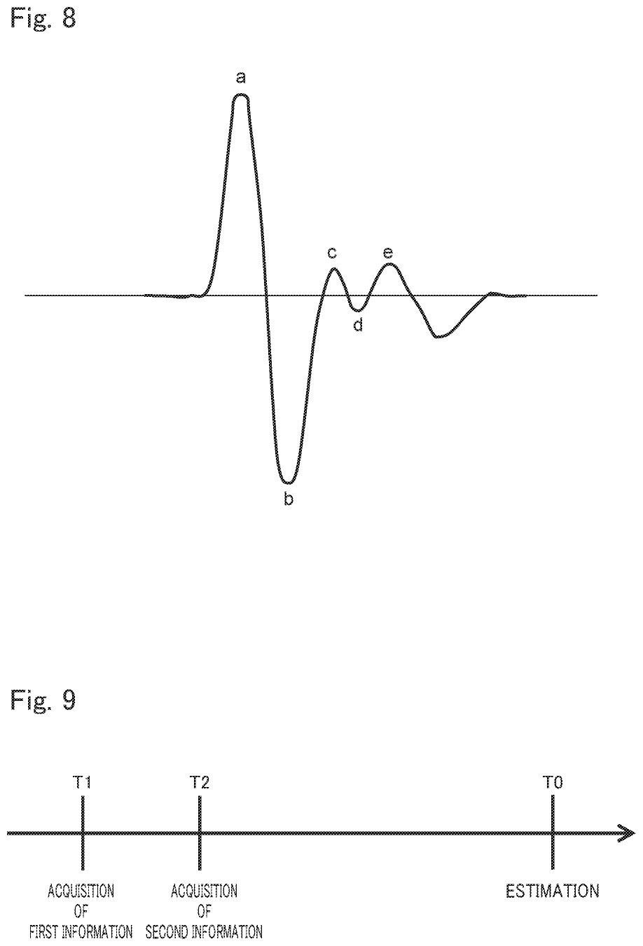

FIG. 8 is a schematic diagram showing an example of an acceleration pulse wave. As shown in FIG. 8, the acceleration pulse wave has an “a wave”, a “b wave”, a “c wave”, a “d wave”, and an “e wave” that peak in chronological order. The peak means a portion where the amplitude increases. In the second embodiment, the ratio between the amplitude of the “a wave” and the amplitude of the “c wave” in the acceleration pulse wave is used as the second information. Specifically, the second information is a value “c/a” obtained by dividing the amplitude value of the “c wave” in the acceleration pulse wave by the amplitude value of the “a wave”.

The second measurement unit 11 transmits the second information to the estimator 20. Alternatively, the second information measured by the second measurement unit 11 is input to the estimator 20. The second measurement unit 11 is controlled by the controller 30.

In the second embodiment, an example in which the acquisition date of the first information and the acquisition date of the second information are different will be described. FIG. 9 is a schematic diagram showing an example of measurement timing. As shown in FIG. 9, the second measurement unit 11 acquires the second information at a timing different from that of the first measurement unit 10. The second measurement unit 11 acquires the second information on a second acquisition date T2 that is a date before the estimation date T0 and is different from the first acquisition date T1 on which the first information is acquired by the first measurement unit 10.

The second acquisition date T2 is a date before the estimation date T0 and after the first acquisition date T1. In the second embodiment, the first acquisition date T1 is a date 10 days before the estimation date T0, and the second acquisition date T2 is 2 days after the first acquisition date T1. In other words, the first acquisition date T1 is a date 10 days before the estimation date, and the second acquisition date T2 is a date 8 days before the estimation date T0.

Similarly to the hormone balance, the blood vessel condition is correlated with the skin condition. In addition, the correlation between the second information related to the blood vessel condition and the skin condition tends to be high on a day before the estimation date T0 and after the first acquisition date T1. Therefore, the second acquisition date T2 on which the second information is acquired is set to a date before the estimation date T0 and after the first acquisition date T1.

The estimator 20 estimates the skin condition in the future from the second acquisition date T2 based on the first information and the second information. In the second embodiment, the estimator 20 has a regression model in which the first information and the second information are input and the information of the future skin condition is output. The estimator 20 estimates the future skin condition by inputting the first information and the second information to the regression model.

In the second embodiment, the estimator 20 estimates the future skin condition on the estimation date T0 on the date of the acquisition date T2 when the second measurement unit 11 acquires the information.

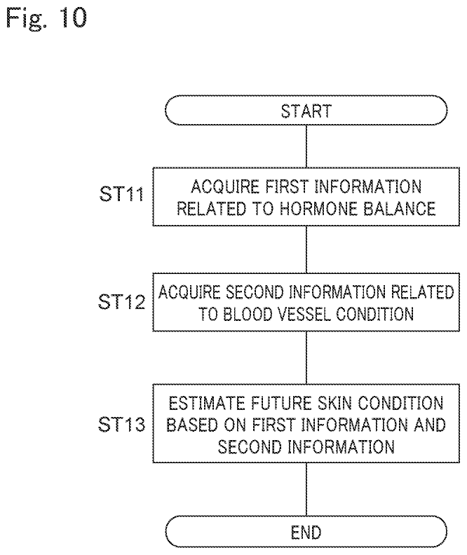

An example of the operation (skin condition estimation method) of the skin condition estimation device 1B will be described with reference to FIG. 10. FIG. 10 is a flowchart showing an example of the skin condition estimation method according to the second embodiment of the present disclosure. In FIG. 10, step ST11 is similar to step ST1 shown in FIG. 4 of the first embodiment, and thus detailed description thereof will be omitted.

As shown in FIG. 10, in step ST11, the first information related to hormone balance is acquired. In step ST11, the first measurement unit 10 acquires the first information.

In step ST12, the second information related to the blood vessel condition is acquired. In step ST12, the second measurement unit 11 acquires the second information on the second acquisition date T2 different from the first acquisition date T1 on which the first information is acquired. The second information includes at least one piece of information of a pulse wave, a blood pressure, and a form and a function of a blood vessel. The second acquisition date T2 is a date before the estimation date T0 and after the first acquisition date T1.

In the second embodiment, in step ST12, an acceleration pulse wave is calculated based on a pulse wave measured by a photoelectric pulse wave sensor. In addition, in step ST12, a value “c/a” obtained by dividing the amplitude value of the “c wave” in the acceleration pulse wave by the amplitude value of the “a wave” is calculated. In step ST12, the calculated value “c/a” is acquired as the second information.

In step ST13, a future skin condition is estimated based on the first information and the second information. In step ST13, the estimator 20 estimates the skin condition in the future from the second acquisition date T2 based on the first information and the second information.

The estimator 20 inputs the first information and the second information to the regression model and executes regression analysis. The estimator 20 inputs the first information acquired on the first acquisition date T1 and the second information acquired on the second acquisition date T2 to the regression model subjected to machine learning in advance. As a result, the estimator 20 estimates the future skin condition on the estimation date T0 after the second acquisition date T2.

### Effects

According to the skin condition estimation method of the second embodiment, the following effects can be obtained.

The skin condition estimation method further includes step ST12 of acquiring the second information related to the blood vessel condition. In step ST13 of estimating, the future skin condition on the estimation date T0 after the second acquisition date T2 when the second information is acquired is estimated based on the first information and the second information. With such a configuration, the future skin condition can be estimated.

The second information includes at least one piece of information of a pulse wave, a blood pressure, and a form and a function of a blood vessel. With such a configuration, the second information related to the blood vessel condition can be easily acquired. Furthermore, the future skin condition can be estimated using the information acquired from the region other than the face. In addition, information such as a pulse wave, a blood pressure, and a form and a function of a blood vessel can be acquired without invading the user.

The second acquisition date T2 on which the second information is acquired is different from the first acquisition date T1 on which the first information is acquired. With such a configuration, the estimation accuracy can be improved by estimating the future skin condition based on the first information and the second information having different acquisition timings.

The second acquisition date T2 is after the first acquisition date T1. With such a configuration, the second information can be acquired at a timing when the correlation between the second information and the skin information becomes high. As a result, the estimation accuracy of the future skin condition can be improved.

The skin condition estimation device 1B includes the first measurement unit 10, the second measurement unit 11, and the estimator 20. The first measurement unit 10 acquires the information related to hormone balance. The second measurement unit 11 acquires the second information related to the blood vessel condition. The estimator 20 estimates the future skin condition on the estimation date T0 after the second acquisition date T2 based on the first information and the second information. With such a configuration, the future skin condition can be estimated.

In the second embodiment, an example in which the first measurement unit 10 and the second measurement unit 11 are separate bodies has been described, but the present disclosure may not be limited thereto. For example, the first measurement unit 10 and the second measurement unit 11 may be integrated.

In the second embodiment, an example in which the second measurement unit 11 measures the second information related to the blood vessel condition has been described, but the present disclosure may not be limited thereto. The second measurement unit 11 only needs to be able to acquire information correlated with the skin condition other than the first information related to hormone balance. The estimator 20 may estimate the future skin condition based on two or more pieces of information correlated with the skin condition. The second information may be subjected to arbitrary processing before being input to the regression model of the estimator 20.

In the second embodiment, an example in which the ratio “c/a” between the “a wave” and the “c wave” calculated based on the acceleration pulse wave is used as the second information has been described, but the present disclosure may not be limited thereto. The second information may be information related to a blood vessel condition. For example, as the second information, a ratio “b/a” between the “a wave” and the “b wave” in the acceleration pulse wave may be used.

In the second embodiment, an example in which the second measurement unit 11 acquires the second information at a timing different from that of the first measurement unit 10 has been described, but the present disclosure may not be limited thereto. The first acquisition date T1 of the first measurement unit 10 and the second acquisition date T2 of the second measurement unit 11 may be the same day. That is, step ST12 of acquiring the second information may acquire the second information on the first acquisition date T1. The estimator 20 may estimate the future skin condition based on the first information and the second information acquired on the same day. Even in such a configuration, the estimation accuracy can be improved.

In the second embodiment, an example in which the first acquisition date T1 is a date 10 days before the estimation date and the second acquisition date T2 is a date 8 days before the estimation date T0 has been described, but the present disclosure may not be limited thereto. The first acquisition date T1 and the second acquisition date T2 may be any date before the estimation date T0.

In the second embodiment, an example in which the skin condition estimation method includes steps ST11 to ST13 has been described, but the skin condition estimation method may not be limited thereto. In the skin condition estimation method, other steps may be added, some steps may be reduced, or a plurality of steps may be performed in one step.

### Third Embodiment

A skin condition estimation method according to a third embodiment of the present disclosure will be described. In the third embodiment, points different from the second embodiment will be mainly described. In the third embodiment, the same or equivalent configurations as those of the second embodiment will be described with the same reference numerals. In the third embodiment, the description overlapping with the second embodiment is omitted.

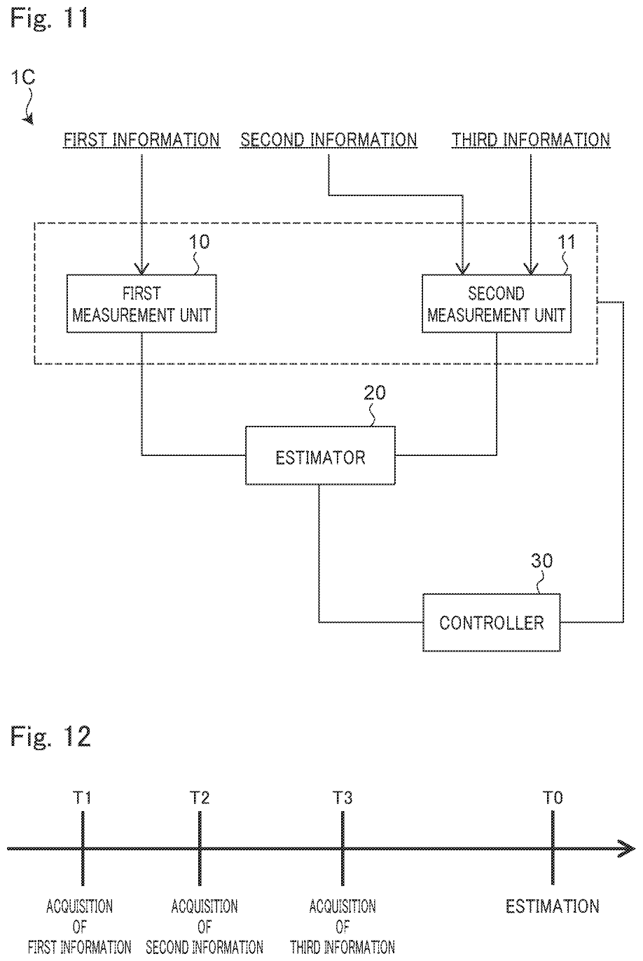

An example of the skin condition estimation method of the third embodiment will be described with reference to FIG. 11. FIG. 11 is a block diagram showing a schematic configuration of an example of a skin condition estimation device 1C according to the third embodiment of the present disclosure.

The third embodiment is different from the second embodiment in that third information related to a blood vessel condition different from the second information is acquired, and the skin condition is estimated based on the first information, the second information, and the third information.

As shown in FIG. 11, the skin condition estimation device 1C acquires the third information by the second measurement unit 11. The third information is information related to a blood vessel condition different from that of the second information. In the third embodiment, the third information is information of the acceleration pulse wave calculated based on the pulse wave measured by the second measurement unit 11, and is a ratio between the amplitude of the “a wave” and the amplitude of the “b wave” in the acceleration pulse wave (see FIG. 8). Specifically, the third information is a value “b/a” obtained by dividing the amplitude value of the “b wave” in the acceleration pulse wave by the amplitude value of the “a wave”.

In the third embodiment, the first information related to hormone balance is information of basal body temperature. The second information related to the blood vessel condition is information of a value “c/a” obtained by dividing the amplitude value of the “c wave” in the acceleration pulse wave by the amplitude value of the “a wave”.

In the third embodiment, an example in which the acquisition date of the first information, the acquisition date of the second information, and the acquisition date of the third information are different from each other will be described. FIG. 12 is a schematic diagram showing an example of measurement timing. As shown in FIG. 12, the third information is acquired on a third acquisition date different from the first acquisition date T1 on which the first information is acquired and the second acquisition date T2 on which the second information is acquired.

The third acquisition date T3 is a date before the estimation date T0 and after the first acquisition date T1. The third acquisition date T3 is a date after the second acquisition date T2. In the third embodiment, the first acquisition date T1 is a date 10 days before the estimation date T0, the second acquisition date T2 is a date 2 days after the first acquisition date T1, and the third acquisition date T3 is a date 7 days after the second acquisition date T2. In other words, the first acquisition date T1 is a date 10 days before the estimation date, the second acquisition date T2 is a date 8 days before the estimation date T0, and the third acquisition date T3 is a date one day before the estimation date T0.

When the third information is a value “b/a” obtained by dividing the amplitude value of the “b wave” in the acceleration pulse wave by the amplitude value of the “a wave”, the correlation between the third information and the skin condition tends to be high on a day before the estimation date T0 and after the first acquisition date T1 and the second acquisition date T2. Therefore, the third acquisition date T3 on which the third information is acquired is set to a date before the estimation date T0 and after the first acquisition date T1 and the second acquisition date T2.

The estimator 20 estimates the skin condition in the future from the third acquisition date T3 based on the first information, the second information, and the third information.

In the third embodiment, the estimator 20 has a regression model in which the first information, the second information, and the third information are input and the information of the future skin condition is output. The estimator 20 estimates the future skin condition by inputting the first information, the second information, and the third information to the regression model.

In the third embodiment, the estimator 20 estimates the future skin condition on the estimation date T0 on the third acquisition date T3 on which the third information is acquired.

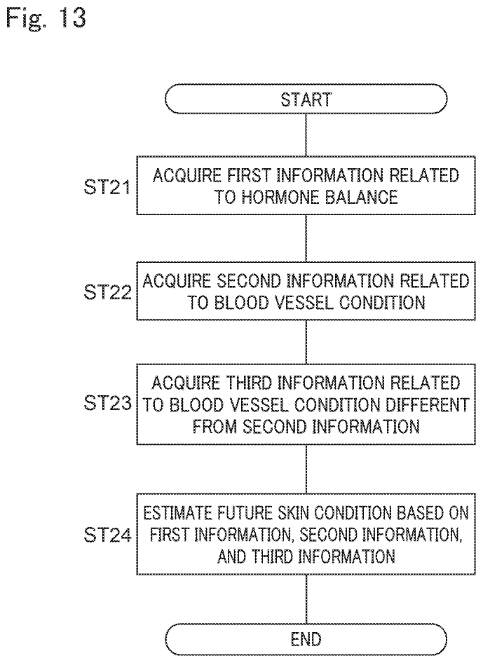

An example of the operation (skin condition estimation method) of the skin condition estimation device 1C will be described with reference to FIG. 13. FIG. 13 is a flowchart showing an example of the skin condition estimation method according to the third embodiment of the present disclosure. In FIG. 13, steps ST21 and ST22 are similar to steps ST11 and ST12 shown in FIG. 10 of the second embodiment, and thus detailed description thereof is omitted.

As shown in FIG. 13, in step ST21, the first information related to hormone balance is acquired. In step ST21, the first measurement unit 10 acquires the first information.

In step ST22, the second information related to the blood vessel condition is acquired. In step ST22, the second measurement unit 11 acquires the second information on the second acquisition date T2 after the first acquisition date T1 on which the first information is acquired.

In step ST23, the third information related to the blood vessel condition different from the second information is acquired. In step ST23, the second measurement unit 1 acquires the third information on the third acquisition date T3 different from the first acquisition date T1 on which the first information is acquired and the second acquisition date T2 on which the second information is acquired. The third acquisition date T3 is a date before the estimation date T0 and after the first acquisition date T1 and the second acquisition date T2.

In the third embodiment, in step ST23, an acceleration pulse wave is calculated based on the pulse wave measured by the second measurement unit 11. In addition, in step ST23, a value “b/a” obtained by dividing the amplitude value of the “b wave” in the acceleration pulse wave by the amplitude value of the “a wave” is calculated. In step ST23, the calculated value “b/a” is acquired as the third information.

In step ST24, a future skin condition is estimated based on the first information, the second information, and the third information. In step ST24, the estimator 20 estimates the skin condition in the future from the third acquisition date T3 based on the first information, the second information, and the third information.

The estimator 20 inputs the first information, the second information, and the third information to the regression model and executes regression analysis. The estimator 20 inputs the first information acquired on the first acquisition date T1, the second information acquired on the second acquisition date T2, and the third information acquired on the third acquisition date T3 to the regression model subjected to machine learning in advance. As a result, the estimator 20 estimates the future skin condition on the estimation date T0 after the third acquisition date T3.

### Effects

According to the skin condition estimation method of the third embodiment, the following effects can be obtained.

The skin condition estimation method further includes step ST23 of acquiring the third information related to the blood vessel condition different from the second information. In step ST24 of estimating, the future skin condition on the estimation date T0 after the third acquisition date T3 is estimated based on the first information, the second information, and the third information. With such a configuration, the future skin condition can be estimated.

The third acquisition date T3 is different from the second acquisition date T2. With such a configuration, the estimation accuracy can be further improved by estimating the future skin condition based on the first information, the second information, and the third information having different acquisition timings.

The third acquisition date T3 is later than the first acquisition date Ti and the second acquisition date T2. With such a configuration, the third information can be acquired at a timing when the correlation between the third information and the skin information becomes high. As a result, the estimation accuracy of the future skin condition can be improved.

In the third embodiment, an example in which the second measurement unit 11 acquires the second information and the third information has been described, but the present disclosure may not be limited thereto. For example, the third information may be acquired by a device different from the second measurement unit 11.

In the third embodiment, an example in which the ratio “b/a” between the “a wave” and the “b wave” calculated based on the acceleration pulse wave is used as the third information has been described, but the present disclosure may not be limited thereto. The third information may be information related to a blood vessel condition. For example, the third information may include at least one piece of information of a pulse wave, a blood pressure, and a form and a function of a blood vessel.

In the third embodiment, an example in which the first acquisition date T1, the second acquisition date T2, and the third acquisition date T3 are different has been described, but the present disclosure may not be limited thereto. For example, at least two of the first acquisition date T1, the second acquisition date T2, and the third acquisition date T3 may be the same date. Even in such a configuration, the estimation accuracy can be improved.

In the third embodiment, an example in which the first acquisition date T1 is a date 10 days before the estimation date, the second acquisition date T2 is a date 8 days before the estimation date T0, and the third acquisition date T3 is a date one day before the estimation date T0 has been described, but the present disclosure may not be limited thereto. For example, the third acquisition date T3 may be a date before the estimation date T0 and before the second acquisition date T2.

In the third embodiment, an example in which the estimator 20 estimates the future skin condition on the estimation date T0 on the third acquisition date T3 has been described, but the present disclosure may not be limited thereto. The estimator 20 may estimate the current skin condition based on the first information, the second information, and the third information acquired in the past.

In the third embodiment, an example in which the skin condition estimation method includes steps ST21 to ST24 has been described, but the skin condition estimation method may not be limited thereto. In the skin condition estimation method, other steps may be added, some steps may be reduced, or a plurality of steps may be performed in one step.

### Second Modification

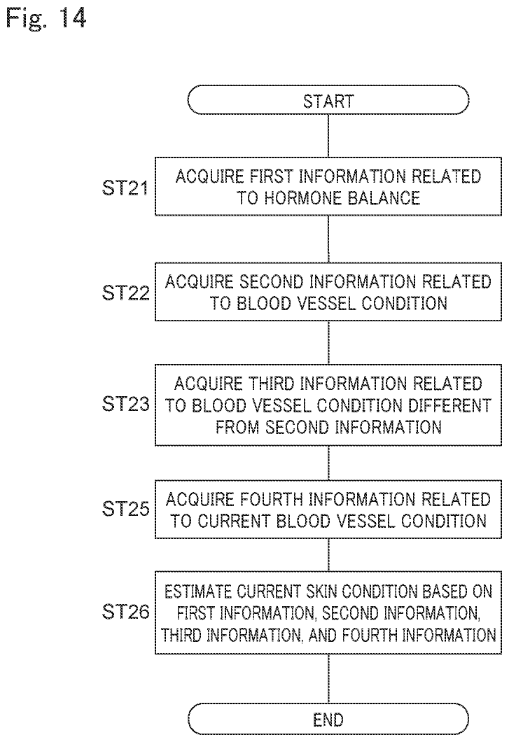

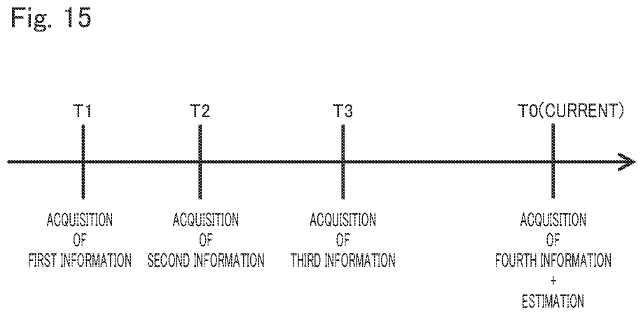

FIG. 14 is a flowchart showing a skin condition estimation method according to a second modification of the third embodiment of the present disclosure. FIG. 15 is a schematic diagram showing an example of measurement timing of the second modification. As shown in FIGS. 14 and 15, the skin condition estimation method of the second modification further includes step ST24 of acquiring fourth information related to a current blood vessel condition, and step ST26 of estimating the current skin condition based on the first information, the second information, the third information, and the fourth information.

In step ST25, the fourth information related to the current blood vessel condition is acquired. The fourth information includes at least one of a pulse wave, a blood pressure, and a form and a function of a blood vessel. In step ST25, the second measurement unit 11 acquires the fourth information. The fourth information is, for example, a ratio “b/a” between the “a wave” and the “b wave” calculated based on the acceleration pulse wave.

In the second modification, the estimation date T0 is current. In step ST25, the second measurement unit 11 acquires the fourth information related to the current blood vessel condition on the estimation date T0.

In step ST26, the current skin condition is estimated based on the first information, the second information, the third information, and the fourth information. In step ST26, the estimator 20 estimates the current skin condition based on the first information, the second information, and the third information acquired in the past and the fourth information acquired at present.

With such a configuration, the current skin condition can be accurately estimated.

In the second modification, an example in which the fourth information is information related to the blood vessel condition has been described, but the present disclosure may not be limited thereto. For example, the fourth information may have at least one of information related to the current hormone balance and information related to the current blood condition.

In the modification 2, an example in which the estimator 20 estimates the current skin condition based on the first information, the second information, the third information, and the fourth information has been described, but the present disclosure may not be limited thereto. For example, the estimator 20 may estimate the current skin condition based on the first information acquired in the past and the fourth information acquired at present. Alternatively, the estimator 20 may estimate the current skin condition based on at least one of the first information, the second information, and the third information acquired in the past and the fourth information acquired currently. Even in such a configuration, the current skin condition can be estimated.

### Fourth Embodiment

A skin condition estimation device and a skin condition estimation method according to a fourth embodiment of the present disclosure will be described. In the fourth embodiment, points different from the third embodiment will be mainly described. In the fourth embodiment, the same or equivalent configurations as those of the third embodiment will be described with the same reference numerals. In the fourth embodiment, the description overlapping with the third embodiment is omitted.

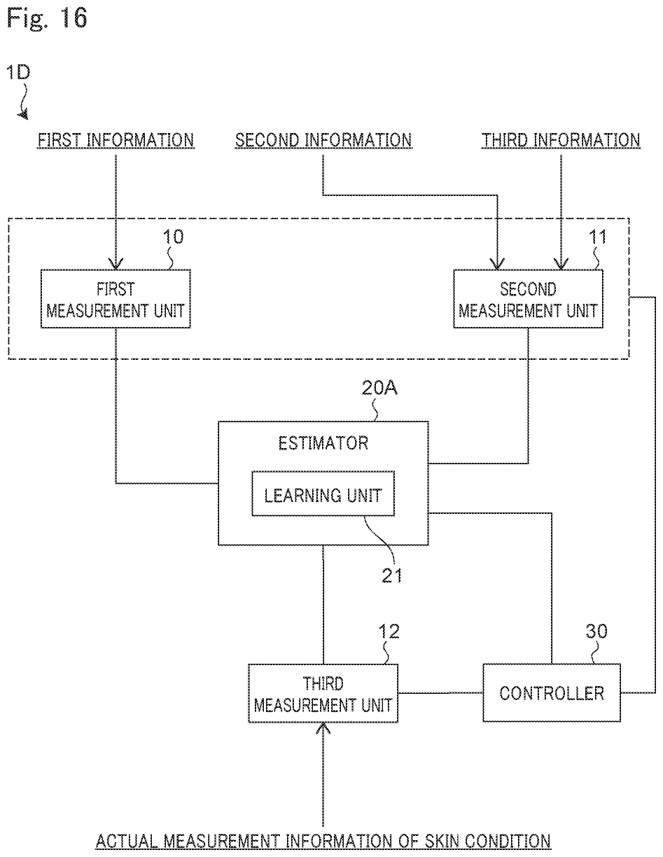

An example of the skin condition estimation device according to the fourth embodiment will be described with reference to FIG. 16. FIG. 16 is a block diagram showing a schematic configuration of an example of a skin condition estimation device 1D according to the fourth embodiment of the present disclosure.

The fourth embodiment is different from the third embodiment in that a third measurement unit 12 that acquires actual measurement information of the skin condition is included and an estimator 20A includes a learning unit 21 (e.g., as part of a machine learning system).

As shown in FIG. 15, the skin condition estimation device 1D includes the third measurement unit 12 that acquires actual measurement information of the skin condition. Furthermore, the estimator 20A includes the learning unit 21.

### Third Measurement Unit

The third measurement unit 12 is a skin measuring instrument that acquires actual measurement information of the skin condition. The third measurement unit 12 acquires, for example, actual measurement information of the skin condition of the face region of the human. The actual measurement information of the skin condition acquired by the third measurement unit 12 is used as teacher data of the learning unit 21 (e.g., training data) described later. The third measurement unit 12 is a device that can quantify the evaluation of the skin condition of the face region of the human. As the third measurement unit 12, for example, a skin analysis system “Beauty Explorer (registered trademark)” manufactured by Sony Corporation can be used. Note that the third measurement unit 12 may not be limited to the skin analysis system “Beauty Explorer (registered trademark)” manufactured by Sony Corporation.

The actual measurement information of the skin condition acquired by the third measurement unit 12 is transmitted to the learning unit 21 of the estimator 20A.

### Learning Unit

The learning unit 21 creates a regression model by machine learning using the first information, the second information, the third information, and the actual measurement information of the skin condition acquired by the third measurement unit 12 as teacher data. Specifically, the learning unit 21 creates a regression model in which the first information, the second information, and the third information are input and the information of the future skin condition is output.

Examples of the machine learning method performed by the learning unit 21 include the k-nearest neighbor algorithm. The k-nearest neighbor algorithm is a method of learning using teacher data, and is a simple method of determining a class label of a sample whose class is unknown by majority decision using neighboring k samples. For example, KNeighborsRegressor registered in the scikit-learn library of Python can be cited.

As the learning result, it is possible to obtain a model with high prediction accuracy by machine learning by selecting the most accurate parameter by the cross test. As a commercially available tool for easily performing the cross test, for example, DataRobot manufactured by DataRobot, Inc. can be used.

Note that the k-nearest neighbor algorithm has been described as the machine learning method performed by the learning unit 21, but the machine learning method may not be limited thereto. For example, the machine learning method performed by the learning unit 21 may be a method using a decision tree or the like.

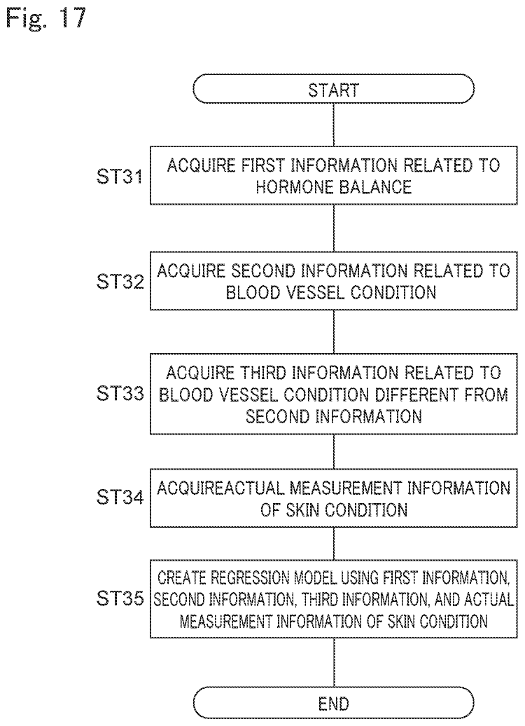

FIG. 17 is a flowchart of an example of a machine learning method in the skin condition estimation method according to the fourth embodiment of the present disclosure. As shown in FIG. 17, in step ST31 of the machine learning method, the first information related to hormone balance is acquired. In step ST31, the first measurement unit 10 acquires the first information. The acquired first information is transmitted to the learning unit 21 of the estimator 20A.

In step ST32, the second information related to the blood vessel condition is acquired. In step ST32, the second measurement unit 11 acquires the second information. The acquired second information is transmitted to the learning unit 21 of the estimator 20A.

In step ST33, the third information related to the blood vessel condition different from the second information is acquired. In step ST33, the second measurement unit 11 acquires the third information. The acquired third information is transmitted to the learning unit 21 of the estimator 20A.

In step ST34, the actual measurement information of the skin condition is acquired. In step ST34, the third measurement unit 12 acquires the actual measurement information of the skin condition. The acquired actual measurement information of the skin condition is transmitted to the learning unit 21 of the estimator 20A.

In step ST35, a regression model is created using the first information, the second information, the third information, and the actual measurement information of the skin condition. In step ST35, the learning unit 21 receives the first information, the second information, the third information, and the actual measurement information of the skin condition. The learning unit 21 creates a regression model using the received first information, second information, third information, and actual measurement information of the skin condition as teacher data. Specifically, the learning unit 21 creates a regression model in which the first information, the second information, and the third information are input and the future skin information is output.

In addition, the learning unit 21 may use the acquisition dates of the first information, the second information, and the third information as teacher data. As a result, the learning unit 21 can calculate the correlation between the acquisition dates of the first information, the second information, and the third information and the skin condition. The learning unit 21 can use the first information, the second information, and the third information acquired on the acquisition date on which the correlation with the skin condition becomes high as inputs to the regression model.

### Effects

According to the skin condition estimation method and the skin condition estimation device according to the fourth embodiment, the following effects can be obtained.

The skin information estimation method includes a machine learning method. The machine learning method includes step ST31 of acquiring the first information, step ST32 of acquiring the second information, step ST33 of acquiring the third information, step ST34 of acquiring the actual measurement information of the skin condition, and step ST35 of creating the regression model. The first information is information related to hormone balance. The second information is information related to a blood vessel condition. The third information is information related to a blood vessel condition different from that of the second information. In step ST35, using the first information, the second information, the third information, and the actual measurement information of the skin condition, a regression model in which the first information, the second information, and the third information are input and a future skin condition is output is created. With such a configuration, it is possible to create a regression model with improved estimation accuracy.

In step ST35, a regression model in which the acquisition dates of the first information, the second information, and the third information are input is further created. With such a configuration, it is possible to estimate the acquisition date on which the correlation with the skin condition becomes high. As a result, it is possible to create a regression model in which the estimation accuracy of the future skin condition is further improved.

The skin condition estimation device 1D includes the third measurement unit 12 and the learning unit 21. The third measurement unit 12 acquires actual measurement information of the skin condition. The learning unit 21 creates a regression model in which the first information, the second information, and the third information are input and a future skin condition is output by machine learning using the first information, the second information, and the third information, and the actual measurement information of the skin condition acquired by the third measurement unit 12 as teacher data. With such a configuration, it is possible to create a regression model with improved estimation accuracy.

In the fourth embodiment, an example in which the skin condition estimation device 1D includes the first measurement unit 10, the second measurement unit 11, and the third measurement unit 12 has been described, but the present disclosure may not be limited thereto. For example, the skin condition estimation device 1D may include the first measurement unit 10 and the third measurement unit 12, and may not include the second measurement unit 11.

Alternatively, in the skin condition estimation device 1D, the measurement units 10 to 12 are not an essential configuration. That is, the skin condition estimation device 1A may not include the measurement units 10 to 12. When the skin condition estimation device 1D does not include the measurement units 10 to 12, the first information, the second information, the third information, and the information of the skin condition may be acquired by a separate measurement device that is not included in the skin condition estimation device 1D. The skin condition estimation device 1D may include an input unit that inputs the first information, the second information, the third information, and the actual measurement information of the skin condition instead of the measurement units 10 to 12. The learning unit 21 may create a regression model using the first information, the second information, the third information, and the actual measurement information of the skin condition input to the input unit as teacher data.

In the fourth embodiment, an example in which the learning unit 21 uses the first information, the second information, and the third information, and the actual measurement information of the skin condition acquired by the third measurement unit 12 as teacher data has been described, but the present disclosure may not be limited thereto. The second information and the third information may not be used as teacher data. In this case, the learning unit 21 may use the first information and the actual measurement information of the skin condition acquired by the third measurement unit 12 as teacher data. That is, the learning unit 21 may create a regression model in which the first information is input and the future skin condition is output using the first information and the actual measurement information of the skin condition as teacher data.

In the fourth embodiment, an example in which the skin condition estimation method includes steps ST31 to ST35 has been described, but the skin condition estimation method may not be limited thereto. In the skin condition estimation method, other steps may be added, some steps may be reduced, or a plurality of steps may be performed in one step.

In the fourth embodiment, an example in which the skin condition estimation method includes the machine learning method has been described, but the present disclosure may not be limited thereto. The machine learning method may not be included in the skin condition estimation method.

In the fourth embodiment, an example in which the estimator 20A is included in the learning unit 21 has been described, but the present disclosure may not be limited thereto. The learning unit 21 may not be included in the estimator 20A. For example, the learning unit 21 may be included in a learning device separate from the skin condition estimation device 1D.

### Fifth Embodiment

A skin condition estimation device according to a fifth embodiment of the present disclosure will be described. In the fifth embodiment, points different from the third embodiment will be mainly described. In the fifth embodiment, the same or equivalent configurations as those of the third embodiment will be described with the same reference numerals. In the fifth embodiment, the description overlapping with the third embodiment is omitted.

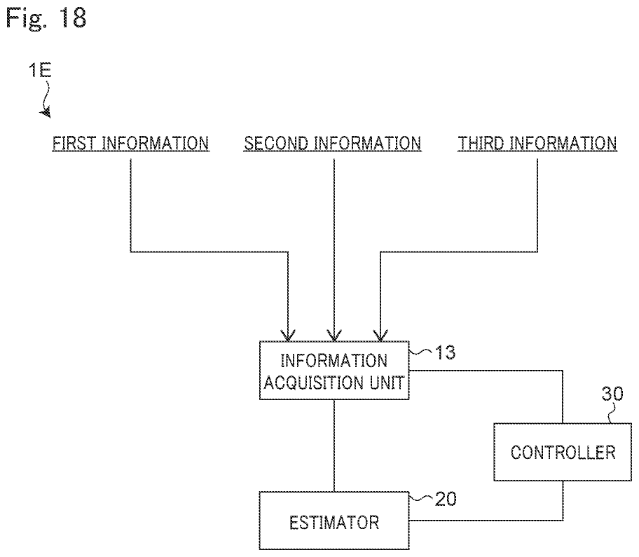

An example of the skin condition estimation device according to the fifth embodiment will be described with reference to FIG. 18. FIG. 18 is a block diagram showing a schematic configuration of an example of a skin condition estimation device 1E according to the fifth embodiment of the present disclosure.

The fifth embodiment is different from the third embodiment in that an information acquisition unit 13 is included instead of the measurement units 10 and 11.

As shown in FIG. 18, the skin condition estimation device 1E includes the information acquisition unit 13, the estimator 20, and the controller 30. In the third embodiment, the measurement units 10 and 11 are devices separate from the skin condition estimation device 1E.

### Information Acquisition Unit

The information acquisition unit 13 acquires the first information, the second information, and the third information. The information acquisition unit 13 is, for example, an input unit that can input information. As the input unit, for example, an input interface such as a keyboard, a mouse, or a touch panel can be used. Alternatively, the input unit may be, for example, a microphone for inputting by voice. The information acquisition unit 13 is controlled by the controller 30.

For example, the user acquires the first information, the second information, and the third information using the first measurement unit 10 and the second measurement unit 11 separate from the skin condition estimation device 1E. For example, the first information, the second information, and the third information are displayed on the display unit of each of the measurement units 10 and 11. The user inputs the first information, the second information, and the third information to the information acquisition unit 13.

The first information, the second information, and the third information input to the information acquisition unit 13 are transmitted to the estimator 20.

The estimator 20 receives the first information, the second information, and the third information from the information acquisition unit 13, and the estimator 20 estimates the future skin condition based on the first information, the second information, and the third information.

### Effects

The skin condition estimation device according to the fifth embodiment can achieve the following effects.

The skin condition estimation device 1E includes the information acquisition unit 13 and the estimator 20. The information acquisition unit 13 acquires the first information, the second information, and the third information. The estimator 20 estimates the future skin condition based on the first information, the second information, and the third information acquired by the information acquisition unit 13. With such a configuration, it is not necessary to include the measurement unit, so that the cost can be reduced.

Note that, in the fifth embodiment, an example in which the information acquisition unit 13 is an input unit capable of inputting information has been described, but the present disclosure may not be limited thereto. For example, the information acquisition unit 13 may include a communicator (e.g., a transmitter and/or receiver) including a circuit that communicates with the measurement units 10, 11, and 12 in conformity with a predetermined communication standard (for example, LAN, Wi-Fi (registered trademark), Bluetooth (registered trademark), USB, HDMI (registered trademark), controller area network (CAN), and serial peripheral interface (SPI)). With such a configuration, information can be easily acquired by receiving information from the measurement units 10 and 11.

In the fifth embodiment, an example in which the information acquisition unit 13 acquires the first information, the second information, and the third information has been described, but the present disclosure may not be limited thereto. The information acquisition unit 13 may acquire at least the first information.

### Sixth Embodiment

A skin condition estimation system and a skin condition estimation method according to a sixth embodiment of the present disclosure will be described. In the sixth embodiment, points different from the first embodiment will be mainly described. In the sixth embodiment, the same or equivalent configurations as those of the first embodiment will be described with the same reference numerals. In the sixth embodiment, the description overlapping with the first embodiment is omitted.

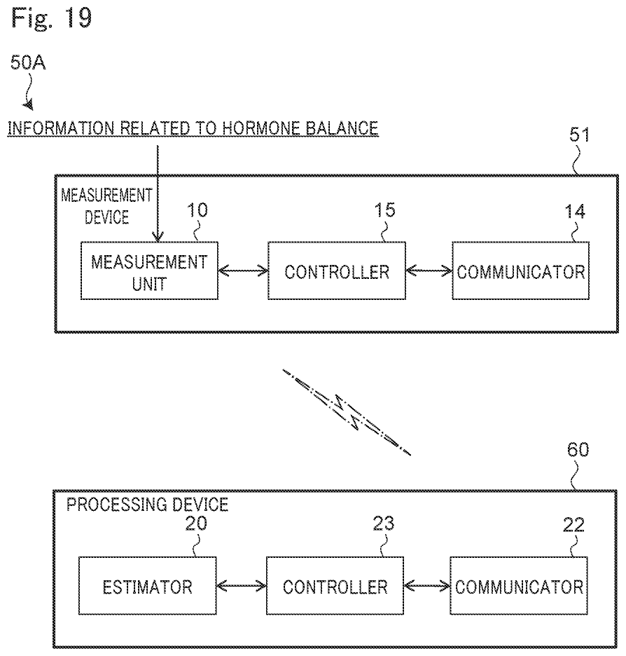

An example of the skin condition estimation system of the sixth embodiment will be described with reference to FIG. 19. FIG. 19 is a block diagram showing a schematic configuration of an example of a skin condition estimation system 50A according to a sixth embodiment of the present disclosure.

As shown in FIG. 19, the skin condition estimation system 50A includes a measurement device 51 and a processing device 60.

### Measurement Device

The measurement device 51 is a device that acquires user information. The measurement device 51 includes the measurement unit 10, a communicator 14, and a controller 15. In the sixth embodiment, the communicator 14 is referred to as a first communicator 14, and the controller 15 is referred to as a first controller 15.

The measurement unit 10 acquires information related to hormone balance. Since the measurement unit 10 is similar to the measurement unit 10 of the first embodiment, detailed description thereof will be omitted.

The first communicator 14 transmits the information acquired by the measurement unit 10. The first communicator 14 includes a circuit that communicates with the processing device 60 in conformity with a predetermined communication standard (for example, LAN, Wi-Fi (registered trademark), Bluetooth (registered trademark), USB, HDMI (registered trademark), controller area network (CAN), and serial peripheral interface (SPI)).

The first controller 15 integrally controls the components of the measurement device 51. The first controller 15 includes, for example, a memory that stores a program, and a processing circuit (not shown) corresponding to a processor such as a central processing unit (CPU). In the first controller 15, the processor executes the program stored in the memory. In the sixth embodiment, the first controller 15 controls the measurement unit 10 and the first communicator 14.

### Processing Device

The processing device 60 is a device that communicates with the measurement device 51. The processing device 60 includes the estimator 20, a communicator 22, and a controller 23. In the sixth embodiment, the communicator 22 is referred to as a second communicator 22, and the controller 23 is referred to as a second controller 23. For example, the processing device 60 is an information processing terminal such as a server.

The estimator 20 estimates the future skin condition on the estimation date after the date on which the information is acquired based on the information related to hormone balance. Since the estimator 20 is similar to the estimator 20 of the first embodiment, detailed description thereof will be omitted.

The second communicator 22 receives information transmitted from the measurement device 51. The second communicator 22 includes a circuit that communicates with the measurement device 51 in conformity with a predetermined communication standard (for example, LAN, Wi-Fi (registered trademark), Bluetooth (registered trademark), USB, HDMI (registered trademark), controller area network (CAN), and serial peripheral interface (SPI)).

The information received by the second communicator 22 is transmitted to the estimator 20.

The second controller 23 integrally controls the components of the processing device 60. The second controller 23 includes, for example, a memory that stores a program, and a processing circuit (not shown) corresponding to a processor such as a central processing unit (CPU). In the second controller 23, the processor executes the program stored in the memory. In the sixth embodiment, the second controller 23 controls the estimator 20 and the second communicator 22.

### Operation

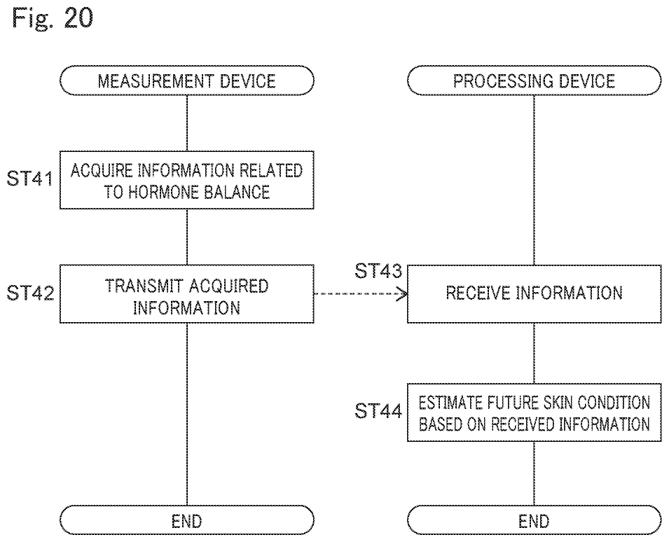

An example of the operation (skin condition estimation method) of the skin condition estimation system 50A will be described with reference to FIG. 20. FIG. 20 is a flowchart showing an example of the skin condition estimation method according to the sixth embodiment of the present disclosure. The skin condition estimation method shown in FIG. 20 is executed by the skin condition estimation system 50A.

As shown in FIG. 20, in step ST41, information related to hormone balance is acquired. In step ST41, the measurement device 51 acquires the information related to hormone balance.

In step ST42, the acquired information related to hormone balance is transmitted. In step ST42, the information acquired by the measurement device 51 is transmitted to the processing device 60.

In step ST43, the transmitted information is received. In step ST43, the processing device 60 receives the information transmitted from the measurement device 51.

In step ST44, a future skin condition is estimated based on the received information. In step ST44, the processing device 60 estimates the future skin condition on the estimation date after the date on which the information is acquired by the measurement device 51 based on the received information. Note that the estimation processing of the estimator 20 is similar to that of the first embodiment, and thus description thereof is omitted.

### Effects

The skin condition estimation system according to the sixth embodiment can achieve the following effects.

The skin condition estimation system 50A includes the measurement device 51 and the processing device 60 that communicates with the measurement device 51. The measurement device 51 includes the measurement unit 10 that acquires information related to hormone balance, and the first communicator 14 that transmits the acquired information. The processing device 60 includes the second communicator 22 that receives information, and the estimator 20 that estimates the future skin condition on the estimation date after the date on which the information is acquired based on the received information. With such a configuration, the future skin condition can be estimated.

In the first embodiment, an example in which the skin condition estimation system 50A includes one measurement device 51 has been described, but the present disclosure may not be limited thereto. The skin condition estimation system 50A may include one or a plurality of measurement devices 51. For example, since the skin condition estimation system 50A includes the plurality of measurement devices 51, estimation accuracy of the skin condition can be improved. In addition, the plurality of measurement devices 51 may acquire different information.

### Third Modification

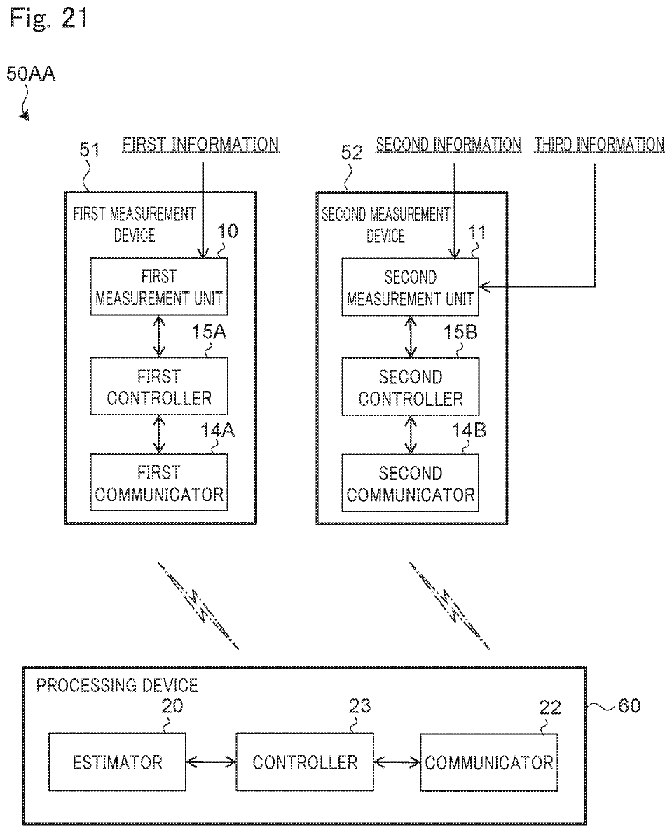

FIG. 21 is a block diagram showing a schematic configuration of an example of a skin condition estimation system 50AA according to a third modification of the sixth embodiment of the present disclosure. As shown in FIG. 21, the skin condition estimation system 50AA includes a plurality of measurement devices 51 and 52. In the skin condition estimation system 50AA, the first measurement device 51 acquires the first information related to hormone balance. The second measurement device 52 acquires the second information and the third information related to the blood vessel information.

The first measurement device 51 includes the first measurement unit 10, a first communicator 14A, and a first controller 15A. In the first measurement device 51, the first measurement unit 10 acquires the first information, and the first communicator 14A transmits the first information to the processing device 60. The first controller 15A controls the first measurement unit 10 and the first communicator 14A.

The second measurement device 52 includes the second measurement unit 11, a second communicator 14B, and a second controller 15B. The second measurement device 52 acquires the second information by the second measurement unit 11, and transmits the second information to the processing device 60 by the second communicator 14B. The second controller 15B controls the second measurement unit 11 and the second communicator 14B.

The processing device 60 receives the first information, the second information, and the third information from the first measurement device 51 and the second measurement device 52. The processing device 60 estimates a future skin condition based on the first information, the second information, and the third information. Note that the estimation processing of the estimator 20 is similar to that of the third embodiment, and thus description thereof is omitted.

Note that, in the third modification, an example in which the skin condition estimation system 50AA includes the two measurement devices 51 and 52 has been described, but the present disclosure may not be limited thereto. The skin condition estimation system 50AA may include a plurality of measurement devices.

### Seventh Embodiment

A skin condition estimation system according to a seventh embodiment of the present disclosure will be described. In the seventh embodiment, points different from the sixth embodiment will be mainly described. In the seventh embodiment, the same or equivalent configurations as those of the sixth embodiment will be described with the same reference numerals. In the seventh embodiment, the description overlapping with the sixth embodiment is omitted.

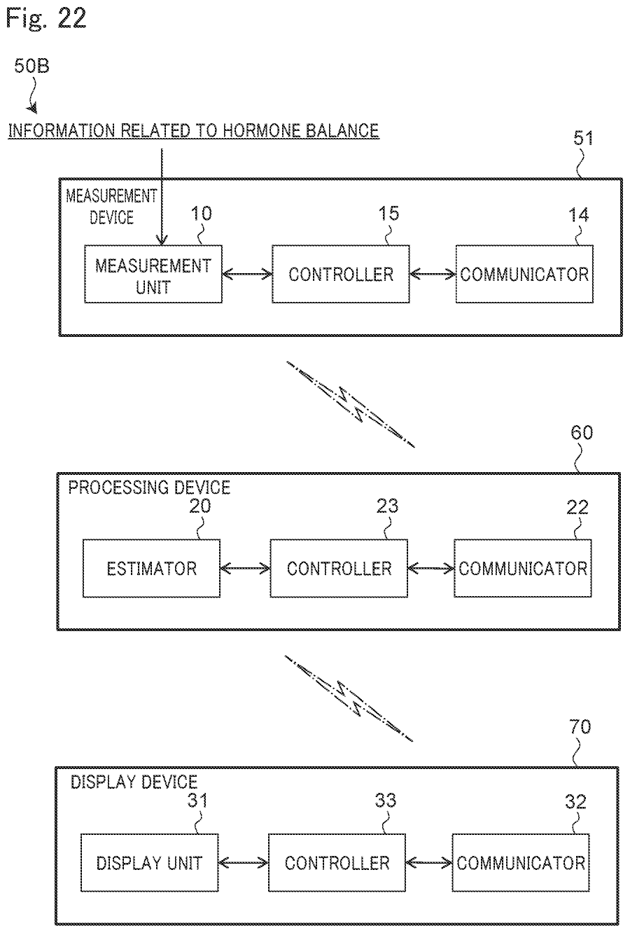

An example of the skin condition estimation system of the seventh embodiment will be described with reference to FIG. 22. FIG. 22 is a block diagram showing a schematic configuration of an example of a skin condition estimation system 50B according to the seventh embodiment of the present disclosure.

The seventh embodiment is different from the sixth embodiment in that a display device 70 is further provided.

As shown in FIG. 22, the skin condition estimation system 50B includes the measurement device 51, the processing device 60, and the display device 70.

### Display Device

The display device 70 is a device that displays the estimation result of the skin condition estimated by the processing device 60. The display device 70 is, for example, an information processing terminal such as a smartphone or an information processing device having a display.

The display device 70 includes the display unit 31, a communicator 32, and a controller 33.

The display unit 31 displays the estimation result of the skin condition estimated by the estimator 20. The display unit 31 is, for example, a display. The display unit 31 is controlled by the controller 33.

The communicator 32 receives the estimation result from the processing device 50.

The controller 33 integrally controls the components of the display device 70. The controller 33 includes, for example, a memory that stores a program, and a processing circuit (not shown) corresponding to a processor such as a central processing unit (CPU). In the controller 33, the processor executes the program stored in the memory. In the seventh embodiment, the controller 33 controls the display unit 31 and the communicator 32.

### Effects

The skin condition estimation system according to the seventh embodiment can achieve the following effects.

The skin condition estimation system 50B further includes the display device 70 that displays the estimation result of the skin condition estimated by the processing device 60. With such a configuration, it is possible to display the estimation result of the future skin condition.

### Eighth Embodiment

A skin condition estimation system according to an eighth embodiment of the present disclosure will be described. In the eighth embodiment, points different from the sixth embodiment will be mainly described. In the eighth embodiment, the same or equivalent configurations as those of the sixth embodiment will be described with the same reference numerals. In the eighth embodiment, the description overlapping with the sixth embodiment is omitted.

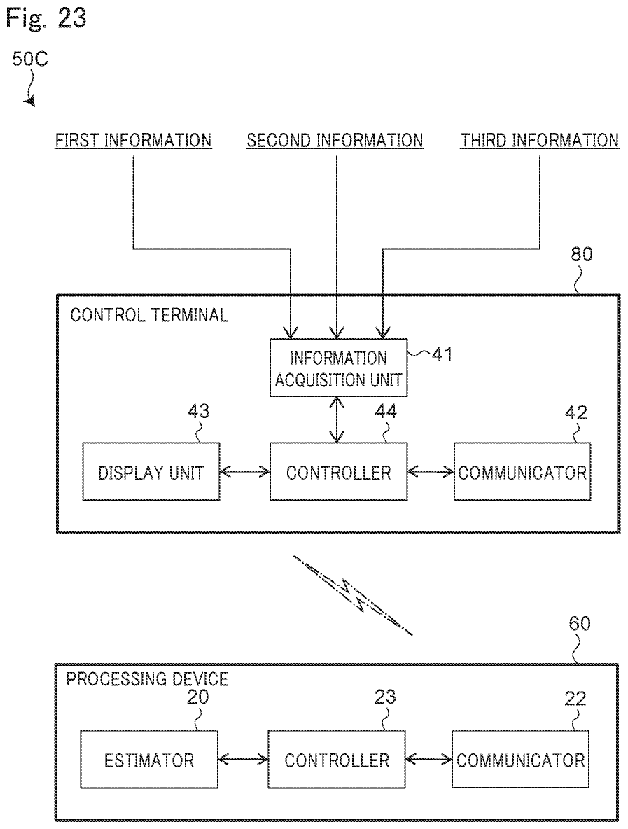

An example of the skin condition estimation system of the eighth embodiment will be described with reference to FIG. 23. FIG. 23 is a block diagram showing a schematic configuration of an example of a skin condition estimation system 50C according to the eighth embodiment of the present disclosure.

The eighth embodiment is different from the sixth embodiment in that a control terminal 80 is further provided.

As shown in FIG. 23, the skin condition estimation system 50C includes the control terminal 80 and the processing device 60.

### Control Terminal

The control terminal 80 acquires the first information, the second information, and the third information, and transmits the first information, the second information, and the third information to the processing device 60. Furthermore, the control terminal 80 receives the estimation result of the skin condition estimated by the processing device 60 from the processing device 60 and displays the estimation result. The control terminal 80 is, for example, an information processing device such as a smartphone or a PC.

The control terminal 80 includes an information acquisition unit 41, a communicator 42, a display unit 43, and a controller 44.

The information acquisition unit 41 acquires the first information, the second information, and the third information. The information acquisition unit 41 is, for example, an input unit that can input information. As the input unit, for example, an input interface such as a keyboard, a mouse, or a touch panel can be used. Alternatively, the input unit may be, for example, a microphone for inputting by voice.

For example, the user inputs the first information, the second information, and the third information acquired by the measurement device to the information acquisition unit 41. The information acquisition unit 41 acquires the information input from the user.

The communicator 42 communicates with the processing device 60. The communicator 42 transmits the first information, the second information, and the third information to the processing device 60. Furthermore, the communicator 42 receives the estimation result of the skin condition from the processing device 60.

The display unit 43 displays the estimation result of the skin condition estimated by the processing device 60. The display unit 43 is, for example, a display.

The controller 44 integrally controls the components of the control terminal 80. The controller 44 includes, for example, a memory that stores a program, and a processing circuit (not shown) corresponding to a processor such as a central processing unit (CPU). In the controller 44, the processor executes the program stored in the memory. In the eighth embodiment, the controller 44 controls the information acquisition unit 41, the communicator 42, and the display unit 43.

### Effects

The skin condition estimation system according to the eighth embodiment can achieve the following effects.

The skin condition estimation system 50C includes the control terminal 80 and the processing device 60. The control terminal 80 acquires the first information, the second information, and the third information, and transmits the first information, the second information, and the third information to the processing device 60. Furthermore, the control terminal 80 receives the estimation result of the skin condition estimated by the processing device 60 and displays the estimation result. The processing device 60 receives the first information, the second information, and the third information from the control terminal 80. The processing device 60 estimates a future skin condition based on the received first information, second information, and third information. The processing device 60 transmits the estimation result of the skin condition to the control terminal 80.

With such a configuration, the control terminal 80 can easily acquire information and display the estimation result of the skin condition. In addition, since the skin condition estimation system 50C does not include the measurement device as an essential component, the cost can be reduced.

Note that, in the eighth embodiment, an example in which the information acquisition unit 41 is an input unit capable of inputting information has been described, but the present disclosure may not be limited thereto. For example, the information acquisition unit 41 may include a communicator including a circuit that communicates with the measurement units 10, 11, and 12 in conformity with a predetermined communication standard (for example, LAN, Wi-Fi (registered trademark), Bluetooth (registered trademark), USB, HDMI (registered trademark), controller area network (CAN), and serial peripheral interface (SPI)). With such a configuration, information can be easily acquired by receiving information from the measurement units 10, 11, and 12.

In the eighth embodiment, an example in which the information acquisition unit 41 acquires the first information, the second information, and the third information has been described, but the present disclosure may not be limited thereto. The information acquisition unit 41 may acquire at least the first information.

In the eighth embodiment, an example in which the control terminal 80 performs both the acquisition of the information and the display of the estimation result of the skin condition has been described, but the present disclosure may not be limited thereto. For example, the control terminal 80 acquires the information, but may not display the estimation result.

### Ninth Embodiment

A skin condition estimation device according to a ninth embodiment of the present disclosure will be described. In the ninth embodiment, points different from the third embodiment will be mainly described. In the ninth embodiment, the same or equivalent configurations as those of the third embodiment will be described with the same reference numerals. In the ninth embodiment, the description overlapping with the third embodiment is omitted.

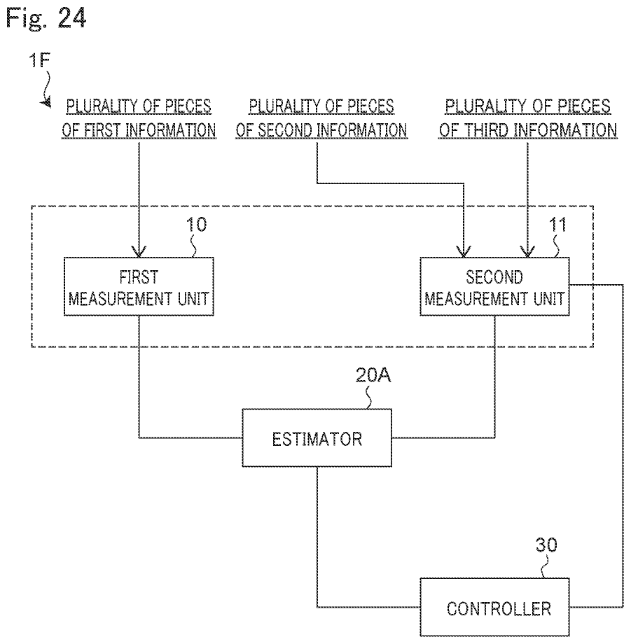

An example of the skin condition estimation device according to the ninth embodiment will be described with reference to FIG. 24. FIG. 24 is a block diagram showing a schematic configuration of an example of the skin condition estimation device 1F according to the ninth embodiment of the present disclosure.

A ninth embodiment is different from the third embodiment in that the first measurement unit 10 and the second measurement unit 11 acquire a plurality of pieces of first information, a plurality of pieces of second information, and a plurality of pieces of third information, and the estimator 20 estimates a future skin condition based on the plurality of pieces of first information, the plurality of pieces of second information, and the plurality of pieces of third information.

As shown in FIG. 24, in the skin condition estimation device 1F, the first measurement unit 10 acquires the plurality of pieces of first information. In addition, the second measurement unit 11 acquires the plurality of pieces of second information and the plurality of pieces of third information.

The first measurement unit 10 acquires the plurality of pieces of first information on a plurality of different days. In the ninth embodiment, the first measurement unit 10 acquires three pieces of first information of 10 days, 9 days, and 8 days before the estimation date T0.

The second measurement unit 11 acquires the plurality of pieces of second information and the plurality of pieces of third information on a plurality of different days. In the ninth embodiment, the second measurement unit 11 acquires three pieces of second information of 10 days, 9 days, and 8 days before the estimation date T0. In addition, the second measurement unit 11 acquires three pieces of third information of 10 days, 9 days, and 8 days before the estimation date T0.

The estimator 20 estimates the future skin condition based on the plurality of pieces of first information, the plurality of pieces of second information, and the plurality of pieces of third information.

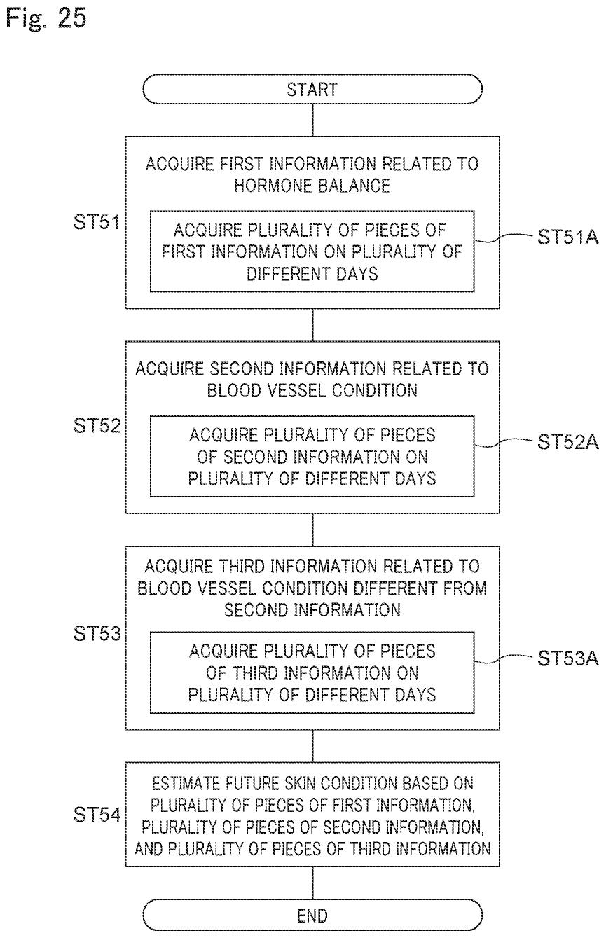

FIG. 25 is a flowchart showing an example of a skin condition estimation method according to a ninth embodiment of the present disclosure. The skin condition estimation method shown in FIG. 25 is executed by the skin condition estimation device 1F.

As shown in FIG. 25, in step ST51, the first information related to hormone balance is acquired. Step ST51 includes step ST51A of acquiring the plurality of pieces of first information on a plurality of different days.

In step ST51A, the first measurement unit 10 acquires the first information a plurality of times on a plurality of different days. For example, the first measurement unit 10 acquires three pieces of first information of 10 days, 9 days, and 8 days before the estimation date T0. In the ninth embodiment, the first information is a basal body temperature.

In step ST52, the second information related to the blood vessel condition is acquired. Step ST52 includes step ST52A of acquiring the plurality of pieces of second information on a plurality of different days.

In step ST52A, the second measurement unit 11 acquires the second information a plurality of times on a plurality of different days. For example, the second measurement unit 11 acquires three pieces of second information of 10 days, 9 days, and 8 days before the estimation date T0. In the ninth embodiment, the second information is a heart beat.

In step ST53, the third information related to the blood vessel condition different from the second information is acquired. Step ST53 includes step ST53A of acquiring the plurality of pieces of third information on a plurality of different days.

In step ST53A, the second measurement unit 11 acquires the third information a plurality of times on a plurality of different days. For example, the second measurement unit 11 acquires three pieces of third information of 10 days, 9 days, and 8 days before the estimation date T0. In the ninth embodiment, the third information is an acceleration pulse wave.

In step ST53, a future skin condition is estimated based on the plurality of pieces of first information, the plurality of pieces of second information, and the plurality of pieces of third information. In step ST53, the estimator 20 estimates the future skin condition based on the plurality of pieces of first information, the plurality of pieces of second information, and the plurality of pieces of third information.

### Effects

According to the skin condition estimation device and the estimation method according to the ninth embodiment, the following effects can be obtained.

In the skin condition estimation device 1F, the first measurement unit 10 acquires the plurality of pieces of first information on a plurality of different days, and the second measurement unit 11 acquires the plurality of pieces of second information and the plurality of pieces of third information on a plurality of different days. The estimator 20 estimates the future skin condition based on the plurality of pieces of first information, the plurality of pieces of second information, and the plurality of pieces of third information.

With such a configuration, the estimation accuracy of the future skin condition can be improved.

In the skin information estimating method, step ST51 of acquiring the first information includes step ST51A of acquiring the plurality of pieces of the first information on a plurality of different days. Step ST52 of acquiring the second information includes step ST52A of acquiring the plurality of pieces of second information on a plurality of different days. Step ST53 of acquiring the third information includes step ST53A of acquiring the plurality of pieces of third information on a plurality of different days. In step ST54 of estimating, the future skin condition is estimated based on the plurality of pieces of first information, the plurality of pieces of second information, and the plurality of pieces of third information.

With such a configuration, the estimation accuracy of the future skin condition can be improved.

In the ninth embodiment, an example in which the plurality of pieces of first information, the plurality of pieces of second information, and the plurality of pieces of third information are three pieces of information acquired by three times of measurement has been described, but the present disclosure may not be limited thereto. The plurality of pieces of first information, the plurality of pieces of second information, and the plurality of pieces of third information may be two or more pieces of information.

In the ninth embodiment, an example in which the plurality of pieces of first information is information measured 10 days before, 9 days before, and 8 days before the estimation date T0 has been described, but the present disclosure may not be limited thereto. For example, the first measurement unit 10 may acquire the first information on a plurality of different days between 7 days or more and 13 days or less before the estimation date T0 on which the skin condition is estimated.

In the ninth embodiment, an example in which the plurality of pieces of second information is information measured 10 days before, 9 days before, and 8 days before the estimation date T0 has been described, but the present disclosure may not be limited thereto. For example, the second measurement unit 11 may acquire the second information 2 days after each day when the first measurement unit 10 acquires the plurality of pieces of first information.

In the ninth embodiment, an example in which the plurality of pieces of third information is information measured 10 days before, 9 days before, and 8 days before the estimation date T0 has been described, but the present disclosure may not be limited thereto. For example, the second measurement unit 11 may acquire the plurality of pieces of third information before the estimation date T0 and after the date on which the plurality of pieces of first information and the plurality of pieces of second information are acquired.

In the ninth embodiment, an example has been described in which the plurality of pieces of first information, the plurality of pieces of second information, and the plurality of pieces of third information are acquired, and the future skin condition is estimated based on the plurality of pieces of first information, the plurality of pieces of second information, and the plurality of pieces of third information, but the present disclosure may not be limited thereto. At least one of the first information, the second information, and the third information may be a plurality of pieces of information. For example, while the first measurement unit 10 performs a plurality of measurements and acquires the plurality of pieces of first information, the second measurement unit 11 may perform one measurement and acquire the second information and the third information. In this case, the estimator 20 may estimate the future skin condition based on the plurality of pieces of first information and the second information and the third information acquired by one measurement.

### Fourth Modification

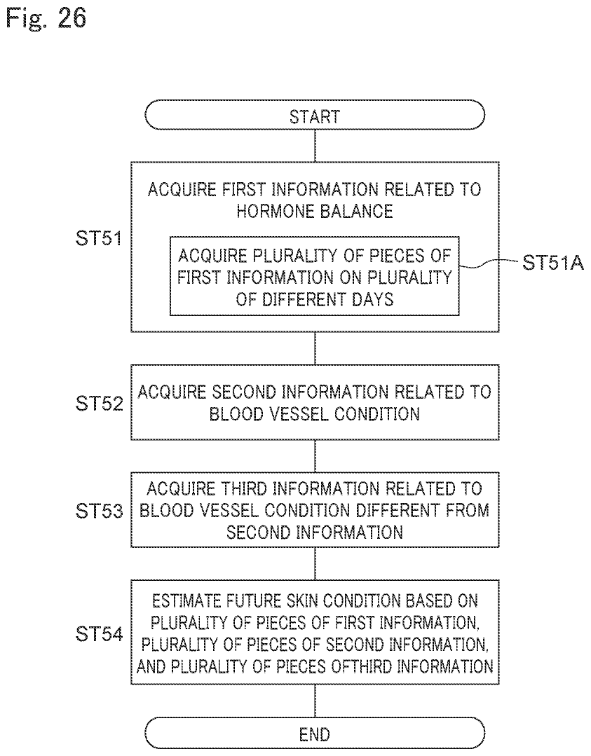

FIG. 26 is a flowchart of a skin condition estimation method according to a fourth modification of the ninth embodiment the present disclosure. As shown in FIG. 26, step ST51 of acquiring the first information includes step ST51A of acquiring the plurality of pieces of first information. On the other hand, step ST52 of acquiring the second information and step ST53 of acquiring the third information may not include step ST52A of acquiring the plurality of pieces of second information and step ST53A of acquiring the plurality of pieces of third information, respectively.

In the fourth modification, in step ST54 of estimating, the future skin condition is estimated based on the plurality of pieces of first information and the second information and the third information acquired by one measurement. Even in such a configuration, the estimation accuracy of the future skin condition can be improved.

In the ninth embodiment, an example in which the future skin condition is estimated based on the plurality of pieces of first information, the plurality of pieces of second information, and the plurality of pieces of third information has been described, but the present disclosure may not be limited thereto. For example, the future skin condition may be estimated based on the plurality of pieces of first information without using the second information and the third information, or the future skin condition may be estimated based on the plurality of pieces of first information and the plurality of pieces of second information without using the third information.

### EXAMPLES

### Example 1 and Example 2

An example 1 and an example 2 will be described.

The example 1 is an estimation result of the skin condition obtained by performing the skin condition estimation method of the first embodiment. In the example 1, the basal body temperature was used as the first information related to hormone balance. The basal body temperature was acquired using a household basal thermometer (MC-652LC manufactured by OMRON Corporation) as the measurement unit 10. In the example 1, the estimator 20 inputs information of the basal body temperature 10 days before the estimation date T0 to the regression model, and estimates the skin condition on the estimation date T0.

The example 2 is an estimation result of the skin condition obtained by performing the skin condition estimation method of the third embodiment. In the example 2, the basal body temperature was used as the first information related to hormone balance. In addition, an acceleration pulse wave, which is information extracted from the pulse wave, was used as the second information and the third information related to the blood vessel information. An optical heart rate sensor was used as the measurement unit 11. The acceleration pulse wave is calculated by secondarily differentiating the measured pulse wave signal. As the second information, a value “c/a” obtained by dividing the amplitude value of the “c wave” in the acceleration pulse wave by the amplitude value of the “a wave” was used. As the third information, a value “b/a” obtained by dividing the amplitude value of the “b wave” in the acceleration pulse wave by the amplitude value of the “a wave” was used. In the example 2, the estimator 20 inputs the first information 10 days before the estimation date T0, the second information 8 days before the estimation date T0, and the third information one day before the estimation date T0 to the regression model, and estimates the skin condition on the estimation date T0.

The measured value is a value acquired by actually measuring the skin condition by a skin measuring instrument. As the skin measuring instrument, a skin analysis system “Beauty Explorer (registered trademark)” manufactured by Sony Corporation was used.

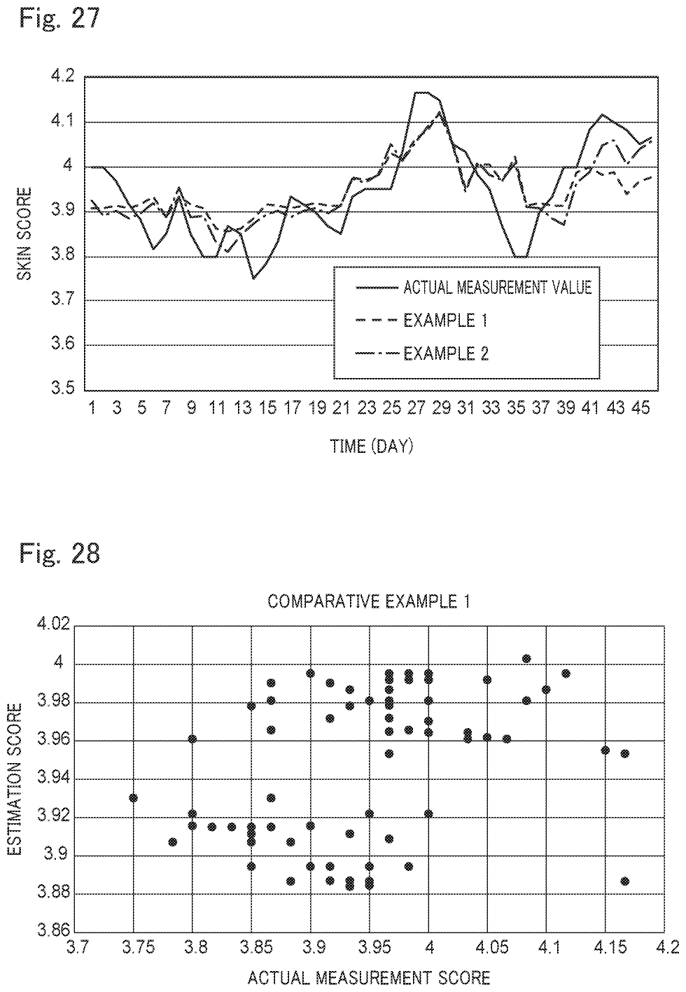

FIG. 27 is a graph showing an example of a correlation between an actual measurement value and the examples 1 and 2. As shown in FIG. 27, it can be seen that the variation tendency of the skin score in the example 1 and the example 2 correlates with the variation tendency of the skin score of the measured value.

The example 1 is a score of the skin condition estimated based on the first information related to hormone balance 10 days before the estimation date T0. From the result shown in FIG. 27, it can be seen that the skin condition 10 days after the acquisition date T1 of the first information can be estimated based on the first information.

The example 2 is a score of the skin condition estimated based on the first information 10 days before the estimation date T0, the second information 8 days before the estimation date T0, and the third information one day before the estimation date T0. From the result shown in FIG. 27, it can be seen that the skin condition 1 day after the acquisition date of the third information can be estimated based on the first to third information. In addition, in the example 2, it can be seen that the future skin condition can be estimated with higher accuracy than in the example 1.

### Comparative Examples 1 to 4

In a comparative example 1, the future skin condition was estimated based on the moisture of the face region. In a comparative example 2, the future skin condition was estimated based on the oil content of the face region. In a comparative example 3, the future skin condition was estimated based on the texture of the face region. In a comparative example 4, the future skin condition was estimated based on the spot on the face region.

In the comparative examples 1 to 4, values measured by a skin measuring instrument were used as the information of moisture, oil content, texture, and spot. As the skin measuring instrument, a skin analysis system “Beauty Explorer (registered trademark)” manufactured by Sony Corporation was used. In the comparative examples 1 to 4, the future skin condition was estimated based on the values measured by the skin measuring instrument.

In the comparative examples 1 to 4, the correlation between the measured value measured by the skin measuring instrument and the estimation value of the skin condition estimated was examined.

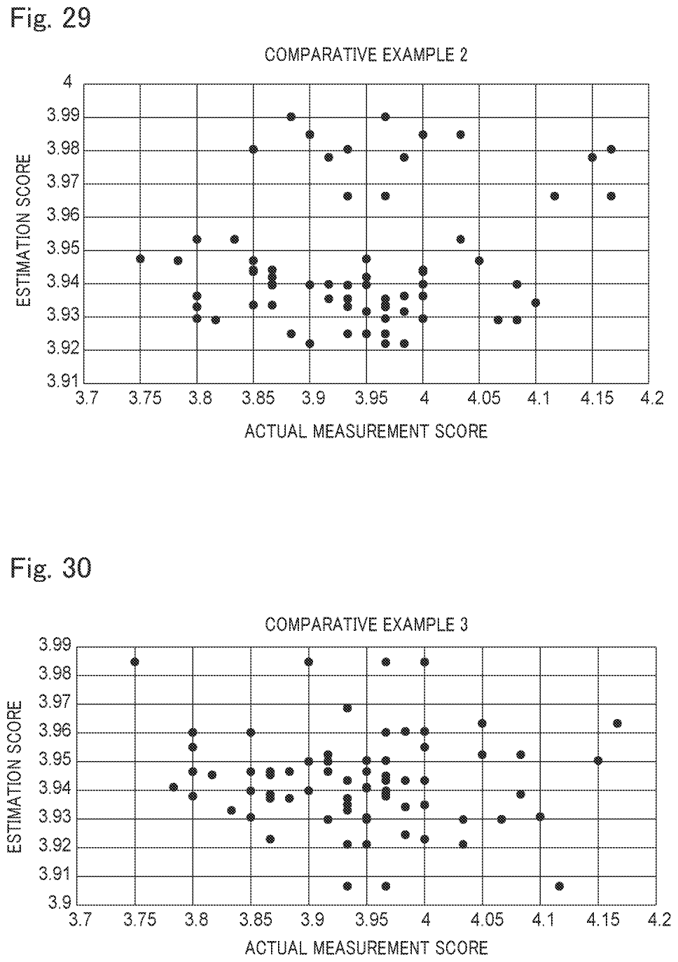

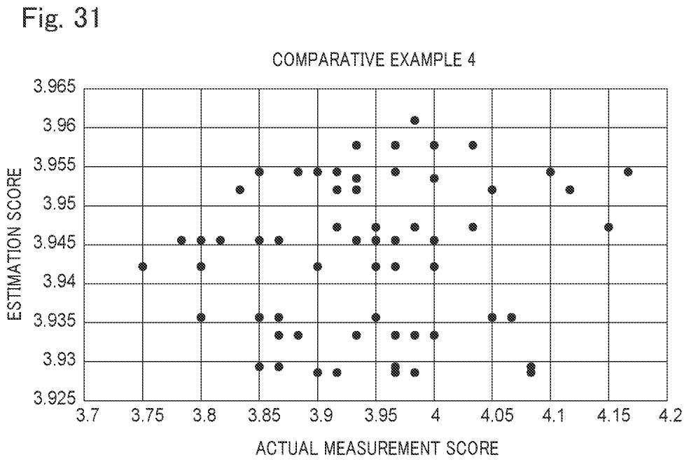

FIG. 28 is a graph showing an example of a correlation between the comparative example 1 and an actual measurement value. FIG. 29 is a graph showing an example of a correlation between the comparative example 2 and an actual measurement value. FIG. 30 is a graph showing an example of a correlation between the comparative example 3 and an actual measurement value. FIG. 31 is a graph showing an example of a correlation between the comparative example 4 and an actual measurement value.

As shown in FIGS. 28 to 31, in the comparative examples 1 to 4, there was no correlation with the measured value. From the results shown in FIGS. 28 to 31, it can be seen that it is difficult to estimate the future skin condition based on the information of the moisture, oil content, texture, and spot of the face region.

### Correlation Coefficients in Examples 1 to 3 and Comparative Examples 1 to 4

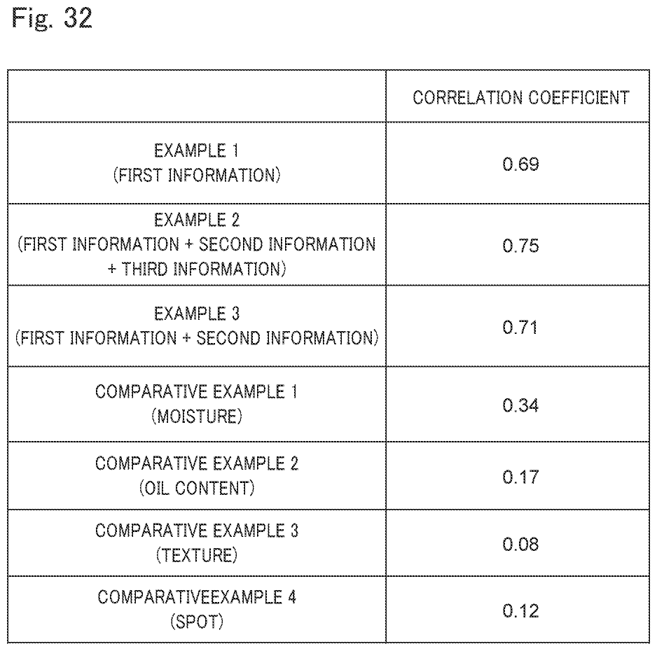

Examples of correlation coefficients of examples 1 to 3 and the comparative examples 1 to 4 will be described with reference to FIG. 32. FIG. 32 is a table showing an example of correlation coefficients of the examples 1 to 3 and the comparative examples 1 to 4.

Note that the example 3 is an estimation result of the skin condition obtained by performing the skin condition estimation method of the second embodiment.

In the example 3, the future skin condition was estimated based on the first information related to hormone balance and the second information related to the blood vessel condition. As the first information, information of basal body temperature was used, and as the second information, a value “c/a” obtained by dividing the amplitude value of the “c wave” in the acceleration pulse wave by the amplitude value of the “a wave” was used. In the example 3, the estimator 20 inputs the first information 10 days before the estimation date T0 and the second information 8 days before the estimation date T0 to the regression model, and estimates the skin condition on the estimation date T0.

As shown in FIG. 32, in the examples 1 to 3, the correlation coefficient is larger than that in the comparative examples 1 to 4, and it can be seen that the correlation coefficient has a correlation with the measured value. In addition, in the example 2 and the example 3, the correlation coefficient exceeds 0.7, and it can be seen that there is a strong correlation.

### Examples 4 to 9

Examples 4 to 9 will be described. In the examples 4 to 9, the first information is a basal body temperature, the second information is a heart beat, and the third information is an acceleration pulse wave.

The example 4 is an estimation result of the skin condition obtained by performing the skin condition estimation method of the first embodiment. In the example 4, the estimator 20 inputs the first information acquired 10 days before the estimation date T0 to the regression model, and estimates the skin condition on the estimation date T0. Note that the example 4 is performed on a day different from that of the example 1.

The example 5 is an estimation result of the skin condition obtained by performing the skin information estimation method of the ninth embodiment. In the example 5, the skin condition on the estimation date T0 was estimated based on the three pieces of first information acquired 10 days, 9 days, and 8 days before the estimation date T0.

The example 6 is an estimation result of the skin condition obtained by performing the skin information estimation method of the second embodiment. In the example 6, the skin condition on the estimation date T0 was estimated based on the first information acquired 10 days before the estimation date T0 and the second information acquired 10 days before the estimation date T0.

The example 7 is an estimation result of the skin condition obtained by performing the skin information estimation method of the ninth embodiment. In the example 7, the skin condition on the estimation date T0 was estimated based on the three pieces of first information acquired 10 days, 9 days, and 8 days before the estimation date T0 and the three pieces of second information acquired 10 days, 9 days, and 8 days before the estimation date T0.

The example 8 is an estimation result of the skin condition obtained by performing the skin information estimation method of the third embodiment. In the example 8, the skin condition on the estimation date T0 was estimated based on the first information acquired 10 days before the estimation date T0, the second information acquired 10 days before the estimation date T0, and the third information acquired 10 days before the estimation date T0.

The example 9 is an estimation result of the skin condition obtained by performing the skin information estimation method of the ninth embodiment. In the example 8, the skin condition on the estimation date T0 was estimated based on the three pieces of first information acquired 10 days, 9 days, and 8 days before the estimation date T0, the three pieces of second information acquired 10 days, 9 days, and 8 days before the estimation date T0, and the three pieces of third information acquired 10 days, 9 days, and 8 days before the estimation date T0.

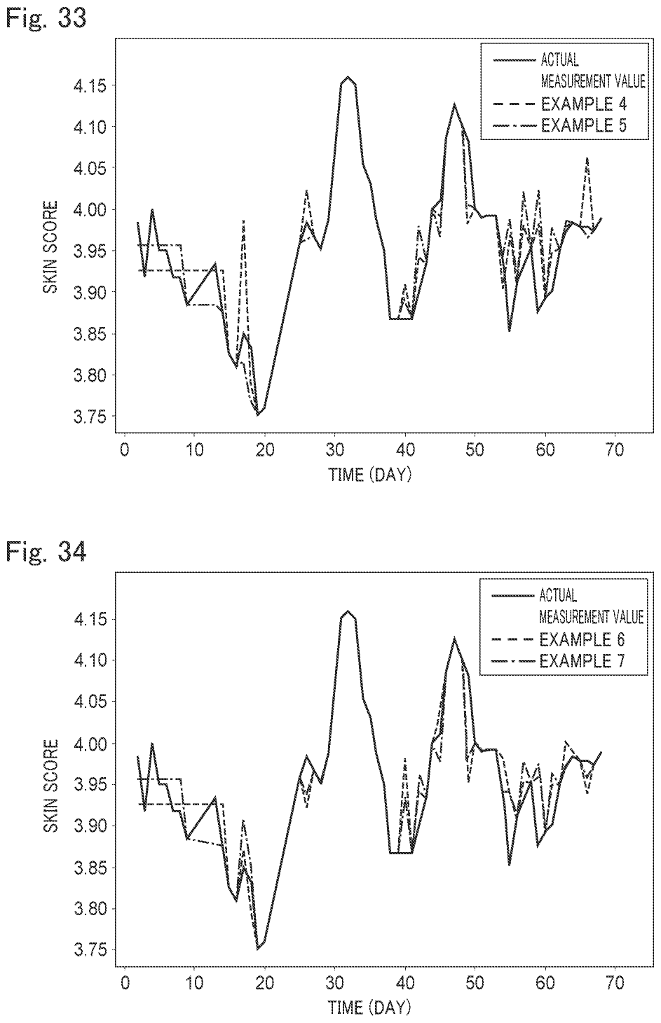

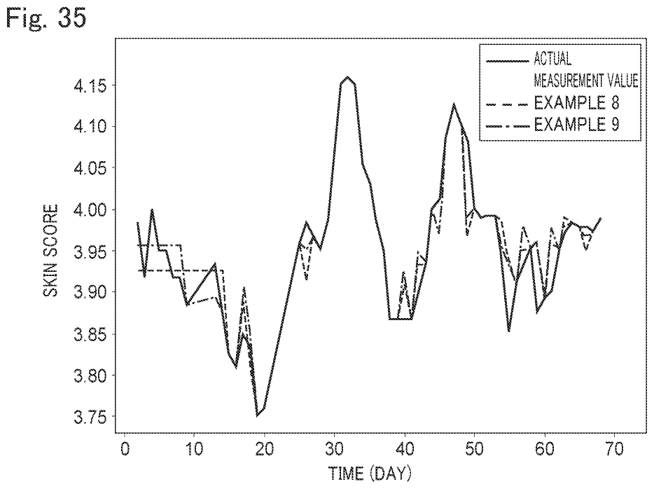

FIG. 33 is a graph showing an example of a correlation between an actual measurement value and the examples 4 and 5. FIG. 34 is a graph showing an example of a correlation between an actual measurement value and the examples 6 and 7. FIG. 35 is a graph showing an example of a correlation between an actual measurement value and the examples 8 and 9. Note that the measured value is a value obtained by actually measuring the skin condition by a skin measuring instrument. As the skin measuring instrument, a skin analysis system Beauty Explorer (registered trademark) manufactured by Sony Corporation was used.

As shown in FIGS. 33 to 35, the estimation results of the skin conditions in the examples 4 to 9 are approximate to the actual measurement values. From these results, it is found that the examples 4 to 9 have a correlation with the measured value.

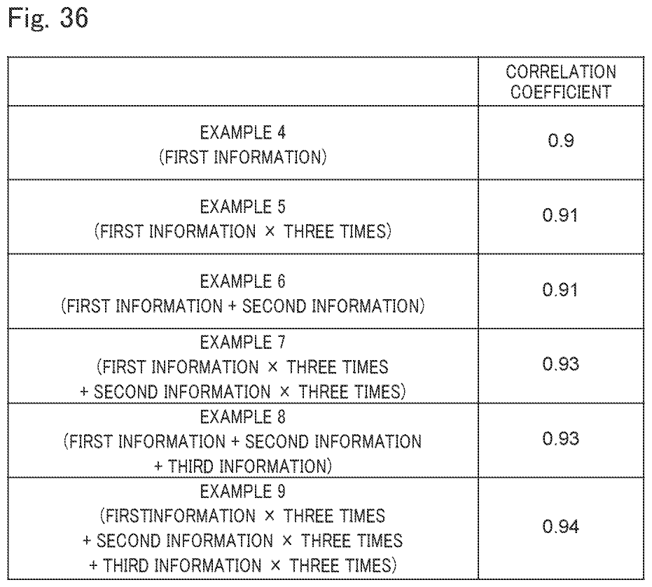

FIG. 36 is a table showing an example of correlation coefficients of the examples 4 to 9. As shown in FIG. 36, a high correlation coefficient is shown in the examples 4 to 9. Furthermore, it can be seen that the correlation coefficient is higher by estimating the skin condition using a plurality of pieces of information or information acquired on a plurality of different days. That is, the estimation accuracy of the skin condition can be further improved by estimating the skin condition using a plurality of pieces of information or information acquired on a plurality of different days.

Note that the regression model used for estimation of the skin condition in the skin condition estimation method according to the present disclosure may be used for cause analysis of a change in the skin condition in a certain period in the past including the present. In a case where the cause of the change in the skin condition in a certain period in the past including the present is analyzed, the change in at least one or more pieces of the already acquired first to third information corresponding to the input of the regression model in the period is independently input to the regression model, and the change value of the skin condition in each information change is obtained. Based on this, the influence of the change in each piece of information of the change in the skin condition can be estimated.

Although the present disclosure has been fully described in connection with preferred embodiments with reference to the accompanying drawings, various modifications and corrections will be apparent to those skilled in the art. Such modifications and corrections are to be understood as being included within the scope of the present disclosure as set forth in the appended claims as long as they do not depart therefrom.

What is claimed is:

1. A skin condition estimation method executed by a computer, the skin condition estimation method comprising:
acquiring first information related to hormone balance; and
estimating a future skin condition on an estimation date, the estimation date being after a first acquisition date, the first acquisition date being a date on which the first information is acquired, and the future skin condition being estimated based on the first information.

2. The skin condition estimation method according to claim 1, wherein the first information comprises at least one piece of information of a basal body temperature, brain waves, blood, saliva, or urine.

3. The skin condition estimation method according to claim 1, wherein the first acquisition date is at least 7 days or more and 13 days or less before the estimation date.

4. The skin condition estimation method according to claim 1,
wherein acquiring the first information comprises acquiring a plurality of pieces of the first information on a plurality of different days, and
wherein the future skin condition is estimated based on the plurality of pieces of the first information acquired on the plurality of different days.

5. The skin condition estimation method according to claim 1, further comprising:
acquiring second information related to a blood vessel condition,
wherein the estimation date is after a second acquisition date on which the second information is acquired, and the future skin condition is estimated based on the first information and the second information.

6. The skin condition estimation method according to claim 5, wherein the second information comprises at least one piece of information of a pulse wave, a blood pressure, or a form and a function of a blood vessel.

7. The skin condition estimation method according to claim 5, wherein the first acquisition date and the second acquisition date are different.

8. The skin condition estimation method according to claim 6, wherein the second acquisition date is later than the first acquisition date.

9. The skin condition estimation method according to claim 5,
wherein acquiring the second information comprises acquiring a plurality of pieces of the second information on a plurality of different days, and
wherein the future skin condition is estimated based on the plurality of pieces of the second information acquired on the plurality of different days.

10. The skin condition estimation method according to claim 5, further comprising:
acquiring third information related to a blood vessel condition different from the second information,
wherein the estimation date is after a third acquisition date on which the third information is acquired, and the future skin condition is estimated based on the first information, the second information, and the third information.

11. The skin condition estimation method according to claim 10, wherein the third acquisition date is different from the second acquisition date.

12. The skin condition estimation method according to claim 11, wherein the third acquisition date is later than the second acquisition date.

13. The skin condition estimation method according to claim 10,
wherein acquiring the third information comprises acquiring a plurality of pieces of the third information on a plurality of different days, and
wherein the future skin condition is estimated based on the plurality of pieces of the third information acquired on the plurality of different days.

14. The skin condition estimation method according to claim 1,
wherein the estimation date is a current day, and
wherein the future skin condition is estimated based on the first information acquired in the past.

15. The skin condition estimation method according to claim 14, further comprising:
acquiring fourth information related to a current hormone balance related to a current blood condition,
wherein estimating the current skin condition comprises estimating the current skin condition based on the first information acquired in the past and the fourth information acquired at present.

16. The skin condition estimation method according to claim 1, further comprising:
acquiring actual measurement information of a skin condition; and
creating a regression model in which the first information is input and the future skin condition is output,
wherein the regression model is trained with the first information and information of the skin condition.

17. The skin condition estimation method according to claim 16, wherein estimating the future skin condition comprises inputting the first information to the regression model.

18. A skin condition estimation device comprising:
a sensor configured to acquire information related to hormone balance; and
at least one processor configured to estimate future skin information on an estimation date, the estimation date being after a date on which the information is acquired, and the future skin information being estimated based on the acquired information.

19. A skin condition estimation system comprising:
a measurement device; and
a processing device configured to communicate with the measurement device,
wherein the measurement device comprises:
a sensor configured to acquire information related to hormone balance; and
a transmitter configured to transmit the information, and
wherein the processing device comprises:
a receiver configured to receive the information; and
at least one processor configured to estimate a future skin condition on an estimation date, the estimation date being after a date on which the information is acquired, and the future skin condition being estimated based on the information.
